SEQUENCE LISTING (24 sequences)

SEQ ID NO: 1 - DNA, 1587 bp, Neurospora crassa
  CDS (1)..(1587)
atggcagagcaagaacacaacggagtcaatggattccattccgagtccgagcagagaaaccaacccgtaaatggtgatgcgagcgaggccgtcaacggaaaccccagcaatggtctcagagtgacgattgaagaaagcgccagcagcgccgtcaacgggggctctcctaccaacagcatgttaacacccatacgacagagaatggaacgcaaaaagtccagtcccatgatgccgacgttcatggtttcggcaccgggaaaagtcattgtgtttggagagcacgcagtcgttcacggcaaggctgcgattgctgccgccatctcgctgcgatcttacctgctcgtcaacacgctttccaagtccaagagaactgttacgctgaaattccctgacatcgacttcaatcattcgtggaatatcgacgagctcccatggaagatcttccaacaaccagggaaaaagaagtactactacagtctcgtcaccgagattgaccaagaactcgttgacgccgtacaacctttcctcgccgatgtctcgatagacaagcccgccgacattcgcaaggtgcaccagaactcggccggctccttcctctacatgttcctttcccttggctcacagtcgttccccggctgccagtacacattgcgctcgacgattcccatcggagccggcctgggcagcagcgcgaccatcgcagtatgcttgtcggcagcgctcttgctccagcttcggacactgtccggtcctcaccccgaccaacctcccgaggaggccaggctacaaattgagcgcatcaaccggtgggcatttgtttacgagatgttcattcacggcaacccctcgggcgtggacaacacagtatcaacacagggcaaggcggtcgtcttccaacggacagactacaaccagccgccctctgtgcgccccctgtgggacttccctaagctcccgctgctcctcgtggacaccaggacggccaagtcaacggcgcacgaggttgccaaggtggccacgctgaagaagaagcacccgcagctggtgggcaccattttgacggccatcgaccaggtcacgcaaagctctgcacagctcattgaggagcaagggttcaacacggaggacgaggagagcctgagcaaggtgggcgagatgatgaccatcaaccacggcctactggtgtcactcggcgtgtcgcacccccgtctggagcgcgtgcgcgagctcgtggaccatgagggtatcgggtggacgaaactcactggtgcgggtggtggcggatgctcgatcacgctgctgcggccgggagtgccgcgcgagaagctggataagctggagcagcgcctggatgaggaggggtactccaagttcgagacaacactaggtagcgacggtgttggcgtactctggccggctgtactgaagaacgggatggacgaggatgaggagggcggtatggagatcgaccttgagaagttcctcagtgcggacagtaacgaggcgcttgagaaacttgtcggtgtacatggcgaccggggcgagcgggagggctggaagttctggcgggttgagaaccgggactag

SEQ ID NO: 2 - protein, 528 aa, Neurospora crassa
MAEQEHNGVNGFHSESEQRNQPVNGDASEAVNGNPSNGLRVTIEESASSAVNGGSPTNSMLTPIRQRMERKKSSPMMPTFMVSAPGKVIVFGEHAVVHGKAAIAAAISLRSYLLVNTLSKSKRTVTLKFPDIDFNHSWNIDELPWKIFQQPGKKKYYYSLVTEIDQELVDAVQPFLADVSIDKPADIRKVHQNSAGSFLYMFLSLGSQSFPGCQYTLRSTIPIGAGLGSSATIAVCLSAALLLQLRTLSGPHPDQPPEEARLQIERINRWAFVYEMFIHGNPSGVDNTVSTQGKAVVFQRTDYNQPPSVRPLWDFPKLPLLLVDTRTAKSTAHEVAKVATLKKKHPQLVGTILTAIDQVTQSSAQLIEEQGFNTEDEESLSKVGEMMTINHGLLVSLGVSHPRLERVRELVDHEGIGWTKLTGAGGGGCSITLLRPGVPREKLDKLEQRLDEEGYSKFETTLGSDGVGVLWPAVLKNGMDEDEEGGMEIDLEKFLSADSNEALEKLVGVHGDRGEREGWKFWRVENRD

SEQ ID NO: 3 - DNA, 759 bp, Fusarium graminearum
  CDS (1)..(759)
gagatgtgtattcatgacaacccttcaggcgtcgacaataccgttgcgacacaaggaaaggctgtggtgtttcaacgaacagattactccaaaccacccaacgttcgcccactgtgggacttccccgaactgcctctattgttggtagacactcgccaggccaagtccactgcacacgaggttgccaaggttgcaaagctgaaacaaacccaccccaagcttgtgaatagcattttagatgctatggataaagtcacagatgctgcttccgaattaatcgaagagacctcttttgataatggatctgtggaggaccttagtaaggttggtgagctgatgaccatcaaccatggcctgttagtatcgctaggagtttcccacccgcgcctggaacgagtacgagagctggtagaccacgggggtattggatggaccaagttgactggcgccggtggtggcggctgctccattacccttctccgccctgatgttcctgctgagaagcttcagaagcttgaagaacgactcgaaaccgaaaattacgccaagtttgagacgacacttggaggtgatggtattggtgtcctctggccagctgttcttaagaacggcaccgaggaagacgaagagggcggcatggagattgatttagagaagttcttagaggctgaaggcacggagggtgtcgagaagctcgttggagtacatggcgatactggggaaagagaaggctggaagttctggagagtggaaagccag

SEQ ID NO: 4 - protein, 253 aa, Fusarium graminearum
EMCIHDNPSGVDNTVATQGKAVVFQRTDYSKPPNVRPLWDFPELPLLLVDTRQAKSTAHEVAKVAKLKQTHPKLVNSILDAMDKVTDAASELIEETSFDNGSVEDLSKVGELMTINHGLLVSLGVSHPRLERVRELVDHGGIGWTKLTGAGGGGCSITLLRPDVPAEKLQKLEERLETENYAKFETTLGGDGIGVLWPAVLKNGTEEDEEGGMEIDLEKFLEAEGTEGVEKLVGVHGDTGEREGWKFWRVESQ

SEQ ID NO: 5 - DNA, 1527 bp, Fusarium graminearum
  CDS (1)..(1527)
atgcctccttcgaacccagccatggttaacgggctcaacggcagccatgccaacggcaacggcaatggtcacaatcatatatctgattctggttcggaaacatctggtgaatcaagcaacggcagcggccgtcgtcgcatgaaactgaaccgcaagatgtccagccctatggcacctcctttcatggtatcggcaccaggaaaggtcattgtttttggagaacactctgttgttcatggcaaggcagccatcgccgcagccatttctctgcggtcatacctacacgttaccaccctttccaagtcgaaacgaaccgtctcgctccgattcgccgatattggtctcgttcacacctggaacatcgaagacctaccgtgggaagcctttcaacagccatccaagaagaagtcgtactattctctcgtgacagagctcgaccccgatctcgtcgccgccattcaaccacacatcgaagttgtctcccccaaccaccccgaggaaatccgaagagtgcgccacagctccgtctccgccttcctatatcttttcttatccctgggatctccttcgttccctccctgtctatacactctccgctcgactatacccattggtgctggcttgggcagcagcgcatcggtttcagtatgcctcgcgtccgcccttcttctacagctacggacgttgtccggcccccacccagatcaacctgcagacgaggctcgacttcaagttgaaaggattaacagatgggcgtttgtgtctgagatgtgtattcatgacaacccttcaggcgtcgacaataccgttgcgacacaaggaaaggctgtggtgtttcaacgaacagattactccaaaccacccaacgttcgcccactgtgggacttccccgaactgcctctattgttggtagacactcgccaggccaagtccactgcacacgaggttgccaaggttgcaaagctgaaacaaacccaccccaagcttgtgaatagcattttagatgctatggataaagtcacagatgctgcttccgaattaatcgaagagacctcttttgataatggatctgtggaggaccttagtaaggttggtgagctgatgaccatcaaccatggcctgttagtatcgctaggagtttcccacccgcgcctggaacgagtacgagagctggtagaccacgggggtattggatggaccaagttgactggcgccggtggtggcggctgctccattacccttctccgccctgatgttcctgctgagaagcttcagaagcttgaagaacgactcgaaaccgaaaattacgccaagtttgagacgacacttggaggtgatggtattggtgtcctctggccagctgttcttaagaacggcaccgaggaagacgaagagggcggcatggagattgatttagagaagttcttagaggctgaaggcacggagggtgtcgagaagctcgttggagtacatggcgatactggggaaagagaaggctggaagttctggagagtggaaagccagtga

SEQ ID NO: 6 - protein, 508 aa, Fusarium graminearum
MPPSNPAMVNGLNGSHANGNGNGHNHISDSGSETSGESSNGSGRRRMKLNRKMSSPMAPPFMVSAPGKVIVFGEHSVVHGKAAIAAAISLRSYLHVTTLSKSKRTVSLRFADIGLVHTWNIEDLPWEAFQQPSKKKSYYSLVTELDPDLVAAIQPHIEVVSPNHPEEIRRVRHSSVSAFLYLFLSLGSPSFPPCLYTLRSTIPIGAGLGSSASVSVCLASALLLQLRTLSGPHPDQPADEARLQVERINRWAFVSEMCIHDNPSGVDNTVATQGKAVVFQRTDYSKPPNVRPLWDFPELPLLLVDTRQAKSTAHEVAKVAKLKQTHPKLVNSILDAMDKVTDAASELIEETSFDNGSVEDLSKVGELMTINHGLLVSLGVSHPRLERVRELVDHGGIGWTKLTGAGGGGCSITLLRPDVPAEKLQKLEERLETENYAKFETTLGGDGIGVLWPAVLKNGTEEDEEGGMEIDLEKFLEAEGTEGVEKLVGVHGDTGEREGWKFWRVESQ

SEQ ID NO: 7 - DNA, 53 bp, Artificial Sequence
  Primer
atgaagcttggggtttgagggccaatggaacgaaactagtgtaccacttgacc

SEQ ID NO: 8 - DNA, 28 bp, Artificial Sequence
  Primer
gacagatctggcgccattcgccattcag

SEQ ID NO: 9 - DNA, 20 bp, Artificial Sequence
  Primer
ggaatcggtcaatacactac

SEQ ID NO: 10 - DNA, 33 bp, Artificial Sequence
  Primer
tgtagatctctattcctttgccctcggacgagt

SEQ ID NO: 11 - DNA, 36 bp, Artificial Sequence
  Primer
ataagaatgcggccgctactccaaaccacccaacgt

SEQ ID NO: 12 - DNA, 32 bp, Artificial Sequence
  Primer
aaatggcgcgcccttctgaagcttctcagcag

SEQ ID NO: 13 - DNA, 34 bp, Artificial Sequence
  Primer
ataagaatgcggccgcaatggccctcgaaacagc

SEQ ID NO: 14 - DNA, 29 bp, Artificial Sequence
  Primer
aaatggcgcgccgcgcccagaatgacacc

SEQ ID NO: 15 - DNA, 22 bp, Artificial Sequence
  Primer
ggggaggaaaggctgtggtgtt

SEQ ID NO: 16 - DNA, 22 bp, Artificial Sequence
  Primer
cgtcttcctcggtgccgttctt

SEQ ID NO: 17 - DNA, 22 bp, Artificial Sequence
  Primer
atgtctccaaaggaagctgagc

SEQ ID NO: 18 - DNA, 22 bp, Artificial Sequence
  Primer
tcgagtgatggatactgcttcg

SEQ ID NO: 19 - DNA, 29 bp, Artificial Sequence
  Primer
cggctacactagaaggacagtatttggta

SEQ ID NO: 20 - DNA, 30 bp, Artificial Sequence
  Primer
gtcaggcaactatggatgaacgaaatagac

SEQ ID NO: 21 - DNA, 18 bp, Artificial Sequence
  Primer
gcagagcaagaacacaac

SEQ ID NO: 22 - DNA, 33 bp, Artificial Sequence
  Primer
ggctactagaagcttctagtcccggttctcaac

SEQ ID NO: 23 - DNA, 24 bp, Artificial Sequence
  Primer
caccatggcagagcaagaacacaa

SEQ ID NO: 24 - DNA, 18 bp, Artificial Sequence
  Primer
gtcccggttctcaacccg

(19) United States
(12) Patent Application Publication
(10) Pub. No.: US 2006/0068393 A1
(43) Pub. Date: Mar. 30, 2006

(54) Mevalonate kinase as a target for fungicides

(76) Inventors: Thierry Lacour, Stutensee (DE); Jan Rether, Kaiserslautern (DE); Annette Freund, Limburgerhof (DE); Ralf-Michael Schmidt, Kirrweiler (DE)

Correspondence Address: HUTCHISON & MASON PLLC, PO BOX 31686, RALEIGH, NC, 27612, US

(21) Appl. No.: 10/544,657

(22) PCT Filed: Jan. 28, 2004
(86) PCT No.: PCT/EP04/00699

§ 371 Date: Aug. 4, 2005

(30) Foreign Application Priority Data
Feb. 5, 2003 (DE) 10304754.9

(51) Int. Cl. C12Q 1/68; C07H 21/04; C12N 9/12; C12N 1/18; C12N 15/74; A01N 37/18; C07K 14/00

(52) U.S. Cl. 435/6; 435/69.3; 435/194; 435/483; 530/300; 514/2; 536/23.7; 435/254.2

(57) ABSTRACT

The present invention relates to the provision of mevalonate kinase as target for fungicides, to the provision of novel nucleic acid sequences, of functional equivalents of the abovementioned nucleic acid sequences and to the use of the gene products of the abovementioned nucleic acid sequences as novel targets for fungicides. Moreover, the present invention relates to methods for identifying fungicides which inhibit a polypeptide with the biological activity of a mevalonate kinase and to the use of these compounds identified via the abovementioned method as fungicides.

The present invention relates to the provision of mevalonate kinase as target for fungicides, to the provision of novel nucleic acid sequences, of functional equivalents of the abovementioned nucleic acid sequences and to the use of the gene products of the abovementioned nucleic acid sequences as novel targets for fungicides. Moreover, the present invention relates to methods for identifying fungicides which inhibit a polypeptide with the biological activity of a mevalonate kinase and to the use of these compounds identified via the abovementioned method as fungicides.

The basic principle of identifying fungicides via the inhibition of a defined target is known (for example U.S. Pat. No. 5,187,071, WO 98/33925, WO 00/77185). In general, there is a great demand for the detection of enzymes which might constitute novel targets for fungicides. Reasons for this, in addition to the resistance problems which arise, include the ongoing endeavor to identify novel fungicidal active ingredients which are distinguished by as wide as possible a spectrum of action, ecological and toxicological acceptability and low application rates.

In practice, the detection of novel targets entails great difficulties since the inhibition of an enzyme which forms part of a metabolic pathway frequently has no further effect on the growth or the infectivity of the pathogenic fungus. This may be attributed to the fact that the pathogenic fungus switches to alternative metabolic pathways whose existence is not known or that the inhibited enzyme is not limiting for the metabolic pathway. The suitability of a gene product as a target can therefore not be predicted, even if the gene function is known.

It is therefore an object of the present invention to identify fungicidal targets and to provide methods which are suitable for identifying fungicidally active compounds.

We have found that this object is achieved by the use of a polypeptide with the biological activity of a mevalonate kinase encoded by a nucleic acid sequence comprising
a) a nucleic acid sequence with the sequence shown in SEQ ID NO:1, or
b) a nucleic acid sequence which, owing to the degeneracy of the genetic code, can be deduced by backtranslation of the amino acid sequences shown in SEQ ID NO:2, or
c) a nucleic acid sequence which, owing to the degeneracy of the genetic code, can be deduced by backtranslation of the amino acid sequence of a functional equivalent of SEQ ID NO:2 which has at least 35% identity with SEQ ID NO:2
   and by the use of proteins encoded by the abovementioned nucleic acid sequences as targets for fungicides.

Some terms used in the description are now defined at this point.

“Affinity tag”: this refers to a peptide or polypeptide whose coding nucleic acid sequence can be fused to the nucleic acid sequence of a polypeptide with the enzymatic, preferably biological, activity of a mevalonate kinase either directly or by means of a linker, using customary cloning techniques. The affinity tag serves for the isolation, concentration and/or selective purification of the recombinant target protein by means of affinity chromatography from total cell extracts. The abovementioned linker can advantageously contain a protease cleavage site (for example for thrombin or factor Xa), whereby the affinity tag can be cleaved from the target protein when required. Examples of common affinity tags are the “His tag”, for example from Qiagen, Hilden, “Strep tag”, the “Myc tag” (Invitrogen, Carlsberg), the tag from New England Biolabs which consists of a chitin-binding domain and an inteine, the maltose-binding protein (pMal) from New England Biolabs, and what is known as the CBD tag from Novagen. In this context, the affinity tag can be attached to the 5′ or the 3′ end of the coding nucleic acid sequence with the sequence encoding the target protein.

“Enzymatic activity/enzyme activity assay”: firstly the term enzymatic activity describes the ability of an enzyme to convert a substrate into a product. The enzymatic activity can be determined in what is known as an activity assay via the increase in the product, the decrease in the substrate (or starting material) or the decrease in a specific cofactor, or via a combination of at least two of the abovementioned parameters, as a function of a defined period of time.

“Expression cassette”: an expression cassette contains a nucleic acid sequence according to the invention linked operably to at least one genetic control element, such as a promoter, and, advantageously, to a further control element, such as a terminator. The nucleic acid sequence of the expression cassette can be for example a genomic or complementary DNA sequence or an RNA sequence, and their semisynthetic or fully synthetic analogs. These sequences can exist in linear or circular form, extrachromosomally or integrated into the genome. The nucleic acid sequences in question can be synthesized or obtained naturally or contain a mixture of synthetic and natural DNA components, or else consist of various heterologous gene segments of various organisms.

Artificial nucleic acid sequences are also suitable in this context as long as they make possible the expression, in a cell or an organism, of a mevalonate kinase. For example, synthetic nucleotide sequences can be generated which have been optimized with regard to the codon usage of the organisms to be transformed.

All of the abovementioned nucleotide sequences can be generated from the nucleotide units by chemical synthesis in a manner known per se, for example by fragment condensation of individual overlapping complementary nucleotide units of the double helix. Oligonucleotides can be synthesized chemically for example in a manner known per se using the phosphoamidite method (Voet, Voet, 2d Edition, Wiley Press New York, pp. 896-897). When preparing an expression cassette, various DNA fragments can be manipulated in such a way that a nucleotide sequence with the correct direction of reading and the correct reading frame is obtained. The nucleic acid fragments are linked with each other via general cloning techniques as are described, for example, in T. Maniatis, E. F. Fritsch and J. Sambrook, “Molecular Cloning: A Laboratory Manual”, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., “Current Protocols in Molecular Biology”, Greene Publishing Assoc. and Wiley-Interscience (1994).

“Operable linkage”: an operable, or functional, linkage is understood as meaning the sequential arrangement of regulatory sequences or genetic control elements in such a way that each of the regulatory sequences, or each of the genetic control elements, can fulfill its intended function when the coding sequence is expressed.

“Functional equivalents” describe, in the present context, nucleic acid sequences which hybridize under standard conditions with SEQ ID NO:1 or parts of SEQ ID NO:1 or SEQ ID NO:2 and which are capable of bringing about the expression of a polypeptide with the enzymatic activity of a mevalonate kinase, preferably with the biological activity of a mevalonate kinase.

To carry out the hybridization, it is advantageous to use short oligonucleotides with a length of approximately 10-50 bp, preferably 15-40 bp, for example of the conserved or other regions, which can be determined in the manner with which the skilled worker is familiar by comparisons with other related genes. However, longer fragments of the nucleic acids according to the invention with a length of 100-500 bp, or the complete sequences, may also be used for hybridization. Depending on the nucleic acid/oligonucleotide used, longer fragment or complete sequence, or depending on which type of nucleic acid DNA or RNA is being used for the hybridization, these standard conditions vary. Thus, for example, the melting temperatures for DNA:DNA hybrids are approximately 10° C. lower than those of DNA:RNA hybrids of the same length.

Standard hybridization conditions are to be understood as meaning, depending on the nucleic acid, for example temperatures of between 42 and 58° C. in an aqueous buffer solution with a concentration of between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. and 65° C., preferably between approximately 30° C. and 45° C. In the case of DNA:RNA hybrids, the hybridization conditions are advantageously 0.1×SSC and temperatures of between approximately 30° C. and 65° C., preferably between approximately 45° C. and 55° C. These hybridization temperatures which have been stated are melting temperature values which have been calculated by way of example for a nucleic acid with a length of approx. 100 nucleotides and a G+C content of 50% in the absence of formamide. The experimental conditions for DNA hybridization are described in relevant textbooks of genetics such as, for example, in Sambrook et al., “Molecular Cloning”, Cold Spring Harbor Laboratory, 1989, and can be calculated using formulae with which the skilled worker is familiar, for example as a function of the length of the nucleic acids, the type of the hybrids or the G+C content. The skilled worker will find further information on hybridization in the following textbooks: Ausubel et al. (eds.), 1985, “Current Protocols in Molecular Biology”, John Wiley & Sons, New York; Hames and Higgins (eds.), 1985, “Nucleic Acids Hybridization: A Practical Approach”, IRL Press at Oxford University Press, Oxford; Brown (ed.), 1991, “Essential Molecular Biology: A Practical Approach”, IRL Press at Oxford University-Press, Oxford.

A functional equivalent is understood as meaning furthermore in particular also natural or artificial mutations of the corresponding nucleic acid sequences of the protein encoded via the nucleic acid sequences according to the invention, and also their homologs from other organisms.

The present invention thus also encompasses, for example, those nucleotide sequences which are-obtained by modification of the nucleic acid sequence of a polypeptide with the enzymatic, preferably biological, activity of a mevalonate kinase.

For example, such modifications can be generated by techniques with which the skilled worker is familiar, such as “Site Directed Mutagenesis”, “Error Prone PCR”, “DNA shuffling” (Nature 370, 1994, pp. 389-391) or “Staggered Extension Process” (Nature Biotechnol. 16, 1998, pp. 258-261). The aim of such a modification can be, for example, the insertion of further cleavage sites for restriction enzymes, the removal of DNA in order to truncate the sequence, the substitution of nucleotides to optimize the codons, or the addition of further sequences. Proteins which are encoded via modified nucleic acid sequences must retain the desired functions despite a deviating nucleic acid sequence.

The term “functional equivalent” can also relate to the amino acid sequence encoded by the nucleic acid sequence in question. In this case, the term “functional equivalent” describes a protein whose amino acid sequence has a defined percentage of identity or homology with the nucleic acid sequence which encodes a polypeptide with the enzymatic, preferably biological, activity of a mevalonate kinase.

Functional equivalents thus comprise naturally occurring variants of the herein-described sequences and artificial nucleic acid sequences, for example those which have been obtained by chemical synthesis and which are adapted to the codon usage, and also the amino acid sequences derived from them.

“Genetic control sequence”: the term “genetic control sequences”, which is to be considered as being equivalent with the term “regulatory sequence”, describes sequences which have an effect on the transcription and, if appropriate, translation of the nucleic acids according to the invention in prokaryotic or eukaryotic organisms. Examples thereof are promoters, terminators or what are known as “enhancer” sequences. In addition to these control sequences, or instead of these sequences, the natural regulation of these sequences may still be present before the actual structural genes and may, if appropriate, have been genetically modified in such a way that the natural regulation has been switched off and the expression of the target gene has been modified, that is to say increased or reduced. The choice of the control sequence depends on the host organism or starting organism. Genetic control sequences furthermore also comprise the 5′-untranslated region, introns or the noncoding 3′ region of genes. Control sequences are furthermore understood as meaning those which make possible homologous recombination or insertion into the genome of a host organism or which permit removal from the genome.

“Homology” or “identity” between two nucleic acid sequences or polypeptide sequences is defined by the identity of the nucleic acid sequence/polypeptide sequence over in each case the entire sequence length, which is calculated by alignment with the aid of the program algorithm GAP (Wisconsin Package Version 10.0, University of Wisconsin, Genetics Computer Group (GCG), Madison, USA), setting the following parameters:

| | |
|---|---|
| Gap Weight: 8 | Length Weight: 2 |
| Average Match: 2,912 | Average Mismatch: −2,003 |

If other parameters for determining identities are used, they will be stated hereinbelow. In the following text, the term “identity” is also used synonymously with the term “homologous” or “homology”.

“Mutations” comprise substitutions, additions, deletions, inversions or insertions of one or more nucleotide residues, which may also bring about changes in the corresponding amino acid sequence of the target protein by substitution, insertion or deletion of one or more amino acids.

“Knock-out transformants” refers to individual cultures of a transgenic organism in which a specific gene has been inactivated selectively via transformation.

“Natural genetic environment” means the natural chromosomal locus in the organism of origin. In the case of a genomic library, the natural genetic environment of the nucleic acid sequence is preferably retained at least in part. The environment flanks the nucleic acid sequence at least at the 5′ or 3′ side and has a sequence length of at least 50 bp, preferably at least 100 bp, especially preferably at least 500 bp, very especially preferably at least 1000 bp, and most preferably at least 5000 bp.

“Polypeptide with the biological activity mevalonate kinase” describes, within the scope of the present invention, a polypeptide whose presence confers the ability to grow and survive in a filamentous fungus, which is . . . by mevalonate kinase, and which is simultaneously capable of catalyzing the reaction catalyzed by a mevalonate kinase obtained from a filamentous fungus, which is the phosphorylation of mevalonate to give 5-phosphomevalonate. If the protein with the biological activity of mevalonate kinase is switched off, the resulting transformants are not viable.

“Polypeptide with the enzymatic activity of a mevalonate kinase” describes an enzyme which is likewise capable of catalyzing the reaction which is catalyzed by a mevalonate kinase derived from a filamentous fungus, which is the phosphorylation of mevalonate to give 5-phosphomevalonate.

Suitable methods for determining the enzymatic activity are described further below.

“Reaction time” refers to the time required for carrying out an enzyme activity assay until a significant finding is obtained; it depends both on the specific activity of the protein employed in the assay and on the method used and the sensitivity of the instruments used. The skilled worker is familiar with the determination of the reaction times. In the case of methods for identifying fungicidally active compounds which are based on photometry, the reaction times are generally between >0 to 360 minutes.

“Recombinant DNA” describes a combination of DNA sequences which can be generated by recombinant DNA technology.

“Recombinant DNA technology”: generally known techniques for fusing DNA sequences (for example described in Sambrook et al., 1989, Cold Spring Harbor, N.Y., Cold Spring Harbor Laboratory Press).

“Replication origins” ensure the multiplication of the expression cassettes or vectors according to the invention in microorganisms and yeasts, for example the pBR322 ori, ColE1 or the P15A ori in E. coli (Sambrook et al.: “Molecular Cloning. A Laboratory Manual”, 2nd ed., Cold Spring Harbor Laboratory Press, Cold Spring Harbor, N.Y., 1989) and the ARS1 ori in yeast (Nucleic Acids Research, 2000, 28(10): 2060-2068).

“Reporter genes” encode readily quantifiable proteins. The transformation efficacy or the expression site or timing can be assessed by means of these genes via growth assay, fluorescence assay, chemoluminescence assay, bioluminescence assay or resistance assay or via a photometric measurement (intrinsic color) or enzyme activity. Very especially preferred in this context are reporter proteins (Schenborn E, Groskreutz D, Mol. Biotechnol. 1999; 13(1):29-44) such as the “green fluorescence protein” (GFP) (Gerdes H H and Kaether C, FEBS Lett. 1996; 389(1):44-47; Chui W L et al., Curr. Biol 1996, 6:325-330; Leffel S M et al., Biotechniques 23(5):912-8, 1997), chloramphenicol acetyl transferase, a luciferase (Giacomin, Plant Sci 1996, 116:59-72; Scikantha, J. Bact. 1996, 178:121; Millar et al., Plant Mol. Biol. Rep. 1992 10:324-414), and luciferase genes, in general β-galactosidase or β-glucuronidase (Jefferson et al., EMBO J. 1987, 6, 3901-3907) or the Ura3 gene.

“Selection markers” confer resistance to antibiotics or other toxic compounds: examples which may be mentioned in this context are the neomycin phosphotransferase gene, which confers resistance to the aminoglycoside antibiotics neomycin (G 418), kanamycin, paromycin (Deshayes A et al., EMBO J. 4 (1985) 2731-2737), the sul gene, which encodes a mutated dihydropteroate synthase (Guerineau F et al., Plant Mol. Biol. 1990; 15(1):127-136), the hygromycin B phosphotransferase gene (Gen Bank Accession NO: K 01193) and the shble resistance gene, which confers resistance to the bleomycin antibiotics such as zeocin. Further examples of selection marker genes are genes which confer resistance to 2-deoxyglucose-6-phosphate (WO 98/45456) or phosphinothricin and the like, or those which confer a resistance to antimetabolites, for example the dhfr gene (Reiss, Plant Physiol. (Life Sci. Adv.) 13 (1994) 142-149). Examples of other genes which are suitable are trpB or hisD (Hartman S C and Mulligan R C, Proc. Natl. Acad. Sci. USA 85 (1988) 8047-8051). Another suitable gene is the mannose phosphate isomerase gene (WO 94/20627), the ODC (ornithine decarboxylase) gene (McConlogue, 1987 in: Current Communications in Molecular Biology, Cold Spring Harbor Laboratory, Ed.) or the Aspergillus terreus deaminase (Tamura K et al., Biosci. Biotechnol. Biochem. 59 (1995) 2336-2338).

“Significant decrease”: based on the activity of the polypeptide encoded via a nucleic acid sequence according to the invention, this is understood as meaning a decrease in the activity of the polypeptide treated with a test compound in comparison with the activity of the polypeptide not incubated with the test compound and which exceeds an error of measurement.

“Target/target protein”: a polypeptide which may take the form of an enzyme in the traditional sense, a structural protein, a protein relevant for developmental processes, transport proteins, regulatory subunits which confer substrate or activity regulation on an enzyme complex. All of the targets or sites of action share the characteristic that the functional presence of the target protein is essential for survival or normal development, growth and/or infectivity of a phytopathogenic organism.

“Transformation” describes a process for introducing heterologous DNA into a pro- or eukaryotic cell. A transformed cell describes not only the product of the transformation process per se, but also all of the transgenic progeny of the transgenic organism generated by the transformation.

“Transgenic”: referring to a nucleic acid sequence, an expression cassette or a vector comprising a nucleic acid sequence according to the invention or an organism transformed with a nucleic acid sequence, expression cassette or vector according to the invention, the term “transgenic” describes all those constructs which have been generated by genetic engineering methods in which either the nucleic acid sequence of the target protein or a genetic control sequence linked operably to the nucleic acid sequence of the target protein or a combination of the abovementioned possibilities are not in their natural genetic environment or have been modified by recombinant methods. In this context, the modification can be achieved, for example, by mutating one or more nucleotide residues of the nucleic acid sequence in question.

Referring to nucleic acid sequences, the term “comprising” or “to comprise” means that the nucleic acid sequence according to the invention may comprise additional nucleic acid sequences at the 3′ and/or at the 5′ terminus, the length of the additional nucleic acid sequences not exceeding 50 bp at the 5′ terminus and 50 bp at the 3′ terminus of the nucleic acid sequences according to the invention, preferably 25 bp at the 5′ and 25 bp at the 3′ terminus, especially preferably 10 bp at the-5′ and 10 bp at the 3′ terminus.

The terpenes are a widespread group of primary and secondary metabolites which are highly diverse in structure and exert very different functions. Sterols, quinones and carotenoids are essential for growth, development and protection from the incidence of light. Secondary metabolites are, for example, mycotoxins such as the trichothecenes, plant growth regulators such as fusicoccin and fungal phytohormones such as, for example, gibberellin (Homann et al. (1996) Curr. Genet. 30, 232-9). All of these compounds consist of a plurality of isoprenoid subunits. Terpenes are formed either by linear combination of the subunits, which leads to geraniol (C10), farnesol (C15), geranylgeraniol (C20), squalene (C30) or similar compounds. Other terpenes are derivatives of these compounds which are formed by cyclization or rearrangement of the subunits. The terpenes are classified as monoterpenes (C10), sesquiterpenes (C15), diterpenes (C20), triterpenes (C30) or sesterpenes (C25) on the basis of the number of isoprenoid units (Herbert, R. M. (1989) Chapman and Hall, New York).

Terpenoid biosynthesis is effected by condensing the C5 precursors isopentyl pyrophosphate (IPP) and dimethylallyl pyrophosphate (DMAPP). In total, two metabolic pathways via which these precursors can be formed are known. In eukaryotes, Archaebacteria and in the cytosol of higher plants, the mevalonate pathway is used. The alternative pathway, known as the non-mevalonate pathway, is found in Eubacteria, green algae and the chloroplasts of higher plants. Fungi have no alternative isoprenoid biosynthesis pathway (Disch, A. and Rohmer, M. (1998) FEMS 168, 201-8).

The biosynthesis via mevalonate is divided into different processes. Initially, the enzymes acetoacetyl-CoA thiolase and 3-hydroxy-3-methylglutaryl-CoA (HMG-CoA) synthase generate HMG-CoA from three acetyl-CoA molecules. Starting from this intermediate, HMG-CoA reductase generates mevalonate in two reduction steps. The mevalonate is phosphorylated by two kinases, viz. mevalonate kinase and phosphomevalonate kinase. 5-Pyrophosphomevalonate is generated. 5-Pyrophosphomevalonate is decarboxylated to give rise to IPP, which is converted into the isomer DMAPP by IPP isomerase.

The Neurospora crassa mevalonate kinase is localized in the cytosol and constitutes a stable homodimer of two 42 kDa subunits. The enzyme requires ATP as cosubstrate and prefers Mg²⁺ over Mn²⁺ (Imblum, R. L. and Rodwell, V. W. (1974) J. Lipid Res.15, 211-22).

The gene encoding mevalonate kinase has been identified in a variety of fungi such as, for example, Neurospora crassa, Saccharomyces cerevisiae and Schizosaccharomyces pombe. Using specific gene knock-out in S. cerevisiae, it has been demonstrated that the protein is essential for this yeast (Oulmouden, A. and Karst, F. (1991) Curr. Genet. 19, 9-14). However, since genes which are known to be essential in S. cerevisiae are not necessarily also essential for filamentous fungi, the results obtained with S. cerevisiae cannot be applied to filamentous fungi.

WO 01/64943 describes an in vivo screening method for identifying substances which inhibit enzymes of the non-mevalonate pathway. The target suitability of enzymes of the mevalonate metabolic pathway is not discussed in this context. U.S. 2002/0119546 Al describes mevalonate kinase as potential target for herbicides. A potential suitability of mevalonate kinase as target for fungicides is not known.

Surprisingly, it has been found that polypeptides with the biological activity of a mevalonate kinase are suitable as targets for fungicides.

The present invention therefore relates to the use of a polypeptide with the enzymatic activity, preferably biological activity, of a mevalonate kinase encoded by a nucleic acid sequence comprising
a) a nucleic acid sequence with the sequence shown in SEQ ID NO:1, or
b) a nucleic acid sequence which, owing to the degeneracy of the genetic code, can be deduced by backtranslation of the amino acid sequences shown in SEQ ID NO:2, or
c) a nucleic acid sequence which, owing to the degeneracy of the genetic code, can be deduced by backtranslation of the amino acid sequence of a functional equivalent of SEQ ID NO:2 which has at least 35% identity with SEQ ID NO:2
   as target for fungicides.

Functional equivalents of the nucleic acid sequences SEQ ID NO:2 as described in c) have at least 35%, 36%, 37%, 38%, 39% or 40%, advantageously 41%, 42%, 43%, 44%, 45%, 46%, 47%, 48%, 49%, 50%, 51%, 52%, 53%, 54%, 55%, 56%, 57%, 58% or 59%, preferably at least 60%, 61%, 62%, 63%, 64%, 65%, 66%, 67%, 68%, 69%, 71%, 72%, 73%, 74%, 75% or 76%, especially preferably at least 77%, 78%, 79%, 80%, 81%, 82%, 83%, 84%, 85%, 86%, 87%, 88%, 89% or 90%, very especially preferably at least 91%, 92%, 93%, 94%, 95%, 96%, 97%, 98% or 99% identity with SEQ ID NO:2.

The abovementioned nucleic acid sequences as described in a) and b) and their functional equivalents as described in c) are derived from a fungus, for example a yeast, such as yeasts of the genus Saccharomyces (S) such as, for example, S. cerevisiae, Schizosaccharomyces such as, for example, Schizosaccharomyces pombe or Pichia such as, for example, P. pastoris, P. methanolica or a filamentous fungus, preferably from a filamentous fungus, especially preferably from a filamentous fungus of the genus Neurospora, Alternaria, Podosphaera, Sclerotinia, Physalospora, Botrytis, Corynespora; Colletotrichum; Diplocarpon; Elsinoe; Diaporthe; Sphaerotheca; Cinula, Cercospora; Erysiphe; Sphaerotheca; Leveillula; Mycosphaerella; Phyllactinia; Gloesporium; Gymnosporangium, Leptotthrydium, Podosphaera; Gloedes; Cladosporium; Phomopsis; Phytopora; Phytophthora; Erysiphe; Fusarium; Verticillium; Glomerella; Drechslera; Bipolaris; Personospora; Phaeoisariopsis; Spaceloma; Pseudocercosporella; Pseudoperonospora; Puccinia; Typhula; Pyricularia; Rhizoctonia; Stachosporium; Uncinula; Ustilago; Gaeumannomyces or Fusarium (F.), very especially preferably from a filamentous fungus selected from the genera and species Neurospora (N.) such as N. crassa, Alternaria, Podosphaera, Sclerotinia, Physalospora, for example, Physalospora canker, Botrytis (B.), for example, B. cinerea, Corynespora, for example, Corynespora melonis; Colletotrichum; Diplocarpon, for example, Diplocarpon rosae; Elsinoe, for example, Elsinoe fawcetti, Diaporthe, for example, Diaporthe citri; Sphaerotheca; Cinula, for example, Cinula neccata, Cercospora; Erysiphe, for example, Erysiphe cichoracearum and Erysiphe graminis; Sphaerotheca, for example, Sphaerotheca fuliginea; Leveillula, for example, Leveillula taurica; Mycosphaerella; Phyllactinia, for example, Phyllactinia kakicola; Gloesporium, for example, Gloesporium kaki; Gymnosporangium, for example, Gymnosporangium yamadae, Leptotthrydium, for example, Leptotthrydium pomi, Podosphaera, for example, Podosphaera leucotricha; Gloedes, for example, Gloedes pomigena; Cladosporium, for example, Cladosporium carpophilum; Phomopsis; Phytopora; Phytophthora, for example, Phytophthora infestans; Verticillium; Glomerella, for example, Glomerella cingulata; Drechslera; Bipolaris; Personospora; Phaeoisariopsis, for example, Phaeoisariopsis vitis; Spaceloma, for example, Spaceloma ampelina; Pseudocercosporella, for example, Pseudocercosporella herpotrichoides; Pseudoperonospora; Puccinia; Typhula; Pyricularia, for example, Pyricularia oryzae; Rhizoctonia; Stachosporium, for example, Stachosporium nodorum; Uncinula, for example, Uncinula necator; Ustilago; Gaeumannomyces (G.) species, for example, G. graminis and Fusarium species, for example, F. dimerium, F. merismoides, F. lateritium, F. decemcellulare, F. poae, F. tricinctum, F. sporotrichioides, F. chlamydosporum, F. moniliforme, F. proliferatum, F. anthophilum, F. subglutinans, F. nygamai, F. oxysporum, F. solani, F. culmorum, F. sambucinum, F. crookwellense, F. avenaceum ssp. avenaceum, F. avenaceum ssp. aywerte, F. avenaceum ssp. nurragi, F. hetrosporum, F. acuminatum ssp. acuminatum, F. acuminatum ssp. armeniacum, F. longipes, F. compactum, F. equiseti, F. scripi, F. polyphialidicum, F. semitectum and F. beomiforme and F. graminearum.

The present invention likewise relates to the use of a polypeptide with the enzymatic, preferably biological, activity of a mevalonate kinase encoded by a nucleic acid sequence comprising
a) a nucleic acid sequence with the sequence shown in SEQ ID NO:5;
b) a functional equivalent which, owing to the degeneracy of the genetic code, can be deduced by backtranslation of the amino acid sequences shown in SEQ ID NO:6; or
c) a functional equivalent which, owing to the degeneracy of the genetic code, can be deduced by backtranslation of the amino acid sequence of a functional equivalent of SEQ ID NO:6 which has at least 35% identity with SEQ ID NO:6;
   as target for fungicides.

Functional equivalents of the nucleic acid sequences SEQ ID NO:6 as described in c) have at least 35%, 36%, 37%, 38%, 39% or 40%, advantageously 41%, 42%, 43%, 44%, 45%, 46%, 47%, 48%, 49%, 50%, 51%, 52%, 53%, 54%, 55%, 56%, 57%, 58% or 59%, preferably at least 60%, 61%, 62%, 63%, 64%, 65%, 66%, 67%, 68%, 69%, 71%, 72%, 73%, 74%, 75% or 76%, especially preferably at least 77%, 78%, 79%, 80%, 81%, 82%, 83%, 84%, 85%, 86%, 87%, 88%, 89% or 90%, very preferably at least 91%, 92%, 93%, 94%, 95%, 96%, 97%, 98% or 99% identity with SEQ ID NO:6.

The abovementioned nucleic acid sequences as described in a) and b) and their functional equivalents as described in c) are derived from a fungicide, for example a yeast or a filamentous fungus, this meaning the abovementioned preferences and genera.

Suitable functional equivalents of SEQ ID NO:1 or SEQ ID NO:5 as described in c) are the nucleic acid sequences from
S. cerevisiae (Gen Bank Acc. No.: X55875)
S. pombe (Gen Bank Acc. No.: AB000541)
N. crassa (Gen Bank Acc. No.: AL513444)
A. thaliana (Gen Bank Acc. No.: X77793)
C. albicans (Gen Bank Acc. No.: ABZ32140)
A. fumigatus (Gen Bank Acc. No.: ABT18664)
Phaffia rhodozyma (Gen Bank Acc. No.: AAZ30173)

The abovementioned sequences are likewise subject-matter of the present invention.
S. cerevisiae (SWISSPROT Acc. No.: P07277)
S. pombe (SWISSPROT Acc. No.: Q09780)
N. crassa (SPTRMBL Acc. No.: Q9C2B7)
C. albicans (GENESEQ_PROT Acc. No.: ABP73590)
A. fumigatus (GENESEQ_PROT Acc. No.: ABJ25364)
A. thaliana (SWISSPROT Acc. No.: P46086)
Phaffia rhodozyma (GENESEQ_PROT Acc. No.: MY43633)

The abovementioned sequences are likewise subject-matter of the present invention.

The functional equivalents as described in c) also encompass a nucleic acid sequence encoding a polypeptide with the biological function of a mevalonate kinase, comprising a nucleic acid sequence which encompasses
(i) a nucleic acid sequence with the sequence shown in SEQ ID NO:3; or
(ii) a nucleic acid sequence which, owing to the degeneracy of the genetic code, can be deduced by backtranslation of the amino acid sequences shown in SEQ ID NO:4; or
(iii) a nucleic acid sequence which, owing to the degeneracy of the genetic code, can be deduced by backtranslating the amino acid sequence of a functional equivalent of SEQ ID NO:4 which has at least 40% identity with SEQ ID NO:4.

Functional equivalents of the nucleic acid sequences SEQ ID NO:4 as described in iii) have at least 40%, 41%, 42%, 43%, 44%, 45%, 46%, 47% or 48%, advantageously 49%, 50%, 51%, 52%, 53%, 54%, 55%, 56%, 57%, 58% or 59%, preferably at least 60%, 61%, 62%, 63%, 64%, 65%, 66%, 67%, 68%, 69%, 71%, 72%, 73%, 74%, 75%, 76%, 77%, 78% or 79%, especially preferably at least 80%, 81%, 82%, 83%, 84%, 85%, 86%, 87%, 88%, 89% or 90%, very especially preferably at least 91%, 92%, 93%, 94%, 95%, 96%, 97%, 98% or 99% identity with SEQ ID NO:4.

The abovementioned nucleic acid sequences as described in i) and ii) and their functional equivalents as described in iii) are derived from a fungus such as a yeast or a filamentous fungus, preferably from a filamentous fungus, with the abovementioned preferences also applying here. However, the genus Fusarium such as, for example, F. dimerium, F. merismoides, F. lateritium, F. decemcellulare, F. poae, F. tricinctum, F. sporotrichioides, F. chiamydosporum, F. moniliforme, F. proliferatum, F. anthophilum, F. subglutinans, F. nygamai, F. oxysporum, F. solani, F. culmorum, F. sambucinum, F. crookwellense, F. avenaceum ssp. avenaceum, F. avenaceum ssp. aywerte, F. avenaceum ssp. nurragi, F. hetrosporum, F. acuminatum ssp. acuminatum, F. acuminatum ssp. armeniacum, F. longipes, F. compactum, F. equiseti, F. scripi, F. polyphialidicum, F. semitectum and Fusarium beomiforme, is especially preferred among the abovementioned genera of filamentous fungi. Within the genus Fusarium, in turn, the fungus F. graminearum is very especially preferred.

Moreover, the present invention claims nucleic acid sequences which encode a protein with the enzymatic, preferably biological, activity of a mevalonate kinase, comprising
a) a nucleic acid sequence with the sequence shown in SEQ ID NO:3, or
b) a nucleic acid sequence which, owing to the degeneracy of the genetic code, can be deduced by backtranslation of the amino acid sequences shown in SEQ ID NO:4, or
c) functional equivalents of SEQ ID NO:3 with at least 70% identity with SEQ ID NO:3,
d) a nucleic acid sequence which, owing to the degeneracy of the genetic code, can be deduced by backtranslation of the amino acid sequence of a functional equivalent of SEQ ID NO:4 which has at least 80% identity with SEQ ID NO:4.

Functional equivalents of the nucleic acid sequences SEQ ID NO:3 have at least 70%, by preference at least 71%, 72%, 73%, 74,% 75%, 76%, 77%, preferably at least 78%, 79%, 80%, 81%, 82%, 83%, 84%, especially preferably at least 85%, 86%, 87%, 88%, 89%, 90%, 91%, 92%, very especially preferably at least 93%, 94%, 95%, 96%, 97%, 98%, 99% identity with SEQ ID NO: 3.

Functional equivalents of the nucleic acid sequences SEQ ID NO:4 have at least 80%, by preference at least 81%, 82%, 83%, 84%, 85%, 86%, 87%, preferably at least 88%, 89%, 90%, 91%, 92%, 93%, especially preferably at least 94%, 95%, 96%, very especially preferably at least 97%, 98%, 99% identity with SEQ ID NO:4.

Also claimed within the context of the present invention are nucleic acid sequences encoding a polypeptide with the enzymatic, preferably biological, activity comprising
a) a nucleic acid sequence with the sequence shown in SEQ ID NO:5; or
b) a nucleic acid sequence which, owing to the degeneracy of the genetic code, can be deduced by backtranslation of the amino acid sequences shown in SEQ ID NO:6; or
c) a functional equivalent of SEQ ID NO:5 which has at least 67% identity with SEQ ID NO:5;
d) a functional equivalent of SEQ ID NO:5 which, owing to the degeneracy of the genetic code, can be deduced by backtranslation of the amino acid sequence of a functional equivalent of SEQ ID NO:6 which has at least 72% identity with SEQ ID NO:6; Functional equivalents of the nucleic acid sequences SEQ ID NO:5 as shown in c) have at least 67%, with preference at least 68%, 69%, 70%, 71%, 72%, 73%, 74% 75%, 76%. or 77%, preferably at least 78%, 79%, 80%, 81%, 82%, 83% or 84%, especially preferably at least 85%, 86%, 87%, 88%, 89%, 90%, 91% or 92%, very especially preferably at least 93%, 94%, 95%, 96%, 97%, 98% or 99% identity with SEQ ID NO:5.

Functional equivalents of the nucleic acid sequences SEQ ID NO:6 have at least 72%, by preference at least 73%, 74% 75%, 76% or 77%, preferably at least 78%, 79%, 80%, 81%, 82%, 83% or 84%, especially preferably at least 85%, 86%, 87%, 88%, 89%, 90%, 91% or 92%, very especially preferably at least 93%, 94%, 95%, 96%, 97%, 98% or 99% identity with SEQ ID NO:6.

SEQ ID NO:1 or SEQ ID NO:3 can be used for generating hybridization probes via which the functional equivalents of the nucleic acid sequences according to the invention, as defined above, may be isolated. Likewise, the full-length clone encompassing SEQ ID NO:3 can be provided via the hybridization probes. The generation of these probes and the experimental procedure are known. For example, this can be effected via the selective generation of radioactive or nonradioactive probes by PCR and the use of suitably labeled oligonucleotides, followed by hybridization experiments. The technologies required for this purpose are detailed, for example, in T. Maniatis, E. F. Fritsch and J. Sambrook, Molecular Cloning: A Laboratory Manual, Cold Spring Harbor Laboratory, Cold Spring Harbor, N.Y. (1989).

The probes in question can furthermore be modified by standard technologies (Lit. SDM or random mutagenesis) in such a way that they can be employed for further purposes, for example, as a probe which hybridizes specifically with mRNA and the corresponding coding sequences for analysis of the corresponding sequences in other organisms. For example, the probe may be used for screening a genomic library or cDNA library of the fungus in question, or in a computer search for analogous sequences in electronic databases.

Other applications of the above-described probes are the analysis of possibly modified expression profiles of the nucleic acid sequences according to the invention in a variety of fungi, by preference phytopathogenic fungi, specifically in connection with certain factors such as an increased resistance to fungicides, the detection of the fungus in plant material and the detection of developing resistances. The term “phytopathogenic fungi” is understood as meaning the following genera and species: Alternaria, Podosphaera, Sclerotinia, Physalospora, for example, Physalospora canker, Botrytis (B.), for example, B. cinerea, Corynespora, for example, Corynespora melonis; Colletotrichum; Diplocarpon, for example, Diplocarpon rosae; Elsinoe, for example, Elsinoe fawcetti, Diaporthe, for example, Diaporthe citri; Sphaerotheca; Cinula, for example, Cinula neccata, Cercospora; Erysiphe, for example, Erysiphe cichoracearum and Erysiphe graminis; Sphaerotheca, for example, Sphaerotheca fuliginea; Leveillula, for example, Leveillula taurica; Mycosphaerella; Phyllactinia, for example, Phyllactinia kakicola; Gloesporium, for example, Gloesporium kaki; Gymnosporangium, for example, Gymnosporangium yamadae, Leptotthrydium, for example, Leptotthrydium pomi, Podosphaera, for example, Podosphaera leucotricha; Gloedes, for example, Gloedes pomigena; Cladosporium, for example, Cladosporium carpophilum; Phomopsis; Phytopora; Phytophthora, for example, Phytophthora infestans; Verticillium; Glomerella, for example, Glomerella cingulata; Drechslera; Bipolaris; Personospora; Phaeoisariopsis, for example, Phaeoisariopsis vitis; Spaceloma, for example, Spaceloma ampelina; Pseudocercosporella, for example, Pseudocercosporella herpotrichbides; Pseudoperonospora; Puccinia; Typhula; Pyricularia, for example, Pyricularia oryzae; Rhizoctonia; Stachosporium, for example, Stachosporium nodorum; Uncinula, for example, Uncinula necator; Ustilago; Gaeumannomyces (G.) species, for example, G. graminis and Fusarium-species, for example, F. dimerium, F. merismoides, F. lateritium, F. decemcellulare, F. poae, F. tricinctum, F. sporotrichioides, F. chlamydosporum, F. moniliforme, F. proliferatum, F. anthophilum, F. subglutinans, F. nygamai, F. oxysporum, F. solani, F. culmorum, F. sambucinum, F. crookwellense, F. avenaceum ssp. avenaceum, F. avenaceum ssp. aywerte, F. avenaceum ssp. nurragi, F. hetrosporum, F. acuminatum ssp. acuminatum, F. acuminatum ssp. armeniacum, F. longipes, F. compactum, F. equiseti, F. scripi, F. polyphialidicum, F. semitectum and F. beomiforme and F. graminearum.

Increased resistance to a fungicide which uses a protein with the biological activity of a mevalonate kinase as target is frequently based on mutation at sites which are essential for substrate specificity, such as, for example, near the active center or at other sites of the protein which affect binding of the substrate. Owing to the modifications described above, binding of the inhibitor, which acts as the fungicide, to the protein with the biological activity of a mevalonate kinase can be made more difficult or indeed prevented, so that a limited fungicidal action, or none at all, is observed in the crops in question.

Since the modifications which occur in this context frequently encompass only a few base pairs, the above-described probes based on the nucleic acid sequences according to the invention or a functional equivalent as described above may be used for detecting suitably mutated nucleic acid sequences according to the invention in fully or partially resistant phytopathogenic fungi, as described above.

After isolation of the corresponding gene or gene segment of the protein with the biological activity of a mevalonate kinase by means of the abovementioned probes, followed by sequencing and comparison with the corresponding wild-type nucleic acid sequence, two methods are available in principle for analytical purposes:
1. Two primer pairs are constructed using customary methods, the first being complementary to the wild-type sequence and the second being complementary to the correspondingly mutated sequence. The correspondingly mutated sequence can now be detected quantitatively and qualitatively via PCR.
2. By modifying the sequence of bases, cleavage sites for restriction enzymes may be generated or disappear. The region in question is amplified by means of flanking primers and subsequently digested with the restriction enzyme(s) in question and/or sequenced, it thus being possible to detect the presence of the mutation in question.

In the following text, nucleic acid sequences comprising
a) a nucleic acid sequence with the sequence shown in SEQ ID NO:1, or
b) a nucleic acid sequence which, owing to the degeneracy of the genetic code, can be deduced by backtranslation of the amino acid sequences shown in SEQ ID NO:2, or
c) a nucleic acid sequence which, owing to the degeneracy of the genetic code, can be deduced by backtranslation of the amino acid sequence of a functional equivalent of SEQ ID NO:2 which has at least 35% identity with SEQ ID NO:2
   will be referred to as “nucleic acid sequences according to the invention”. Likewise, the term “nucleic acid sequences according to the invention” is understood as meaning nucleic acid sequences comprising
a) a nucleic acid sequence with the sequence shown in SEQ ID NO:5;
b) a functional equivalent which, owing to the degeneracy of the genetic code, can be deduced by backtranslation of the amino acid sequences shown in SEQ ID NO:6; or
c) a functional equivalent which, owing to the degeneracy of the genetic code, can be deduced by backtranslation of the amino acid sequence of a functional equivalent of SEQ ID NO:6 which has at least 35% identity with SEQ ID NO:6.

The term “nucleic acid sequences according to the invention” also encompasses the following nucleic acid sequences, which constitute embodiments of the abovementioned nucleic acid sequence c) and which encompass a nucleic acid sequence comprising a nucleic acid sequence comprising
i) a nucleic acid sequence with the sequence shown in SEQ ID NO:3; or
ii) a nucleic acid sequence which, owing to the degeneracy of the genetic code, can be deduced by backtranslation of the amino acid sequences shown in SEQ ID NO:4; or
iii) a nucleic acid sequence which, owing to the degeneracy of the genetic code, can be deduced by backtranslating the amino acid sequence of a functional equivalent of SEQ ID NO:4 which has at least 40% identity with SEQ ID NO:4.

A polypeptide encoded by a nucleic acid according to the invention with the enzymatic, preferably biological, activity of a mevalonate kinase is hereinbelow referred to as “MEK”. A polypeptide with the enzymatic, preferably biological, activity of a mevalonate kinase is hereinbelow referred to as mevalonate kinase.

The present invention furthermore relates to expression cassettes comprising
a) genetic control sequences in operable linkage with a nucleic acid sequence comprising a nucleic acid sequence encompassing
   i) a nucleic acid sequence with the sequence shown in SEQ ID NO:3, or
   ii) a nucleic acid sequence which, owing to the degeneracy of the genetic code, can be deduced by backtranslation of the amino acid sequences shown in SEQ ID NO:4, or
   iii) functional equivalents of SEQ ID NO:3 with at least 70% identity with SEQ ID NO:3,
   iv) a nucleic acid sequence which, owing to the degeneracy of the genetic code, can be deduced by backtranslation of the amino acid sequence of a functional equivalent of SEQ ID NO:4 which has at least 80% identity with SEQ ID NO:4; or
b) additional functional elements; or
c) a combination of a) and b).

A further subject matter of the invention are expression cassettes comprising
a) genetic control sequences in operable linkage with a nucleic acid sequence comprising
   i) a nucleic acid sequence with the sequence shown in SEQ ID NO:5; or
   ii) a nucleic acid sequence which, owing to the degeneracy of the genetic code, can be deduced by backtranslation of the amino acid sequences shown in SEQ ID NO:6; or
   iii) a functional equivalent of SEQ ID NO:5 with at least 67% identity to SEQ ID NO:5;
   iv) a functional equivalent of SEQ ID NO:5 which, owing to the degeneracy of the genetic code, can be deduced by backtranslation of the amino acid sequence of a functional equivalent of SEQ ID NO:6 which has at least 72% identity to SEQ ID NO:6.
or
b) additional functional elements; or
c) a combination of a) and b).

Vectors comprising the abovementioned expression cassette and the use of expression cassettes comprising
a) genetic control sequences in operable linkage with a nucleic acid sequence according to the invention, or
b) additional functional elements, or
c) a combination of a) and b)
   and the use of vectors comprising the two embodiments of the abovementioned expression cassettes in “in vitro” or “in vivo” test systems.

A further subject matter is the use of the abovementioned embodiments of the expression cassettes (hereinbelow referred to as “expression cassettes according to the invention”) for the expression of MEK for in vitro or in vivo test systems.

In a preferred embodiment, an expression cassette according to the invention comprises a promoter at the 5′ end of the coding sequence and, at the 3′ end, a transcription termination signal and, if appropriate, further genetic control sequences which are linked operably with the interposed coding sequence for the MEK gene.

Equivalents of the above-described expression cassettes which can be brought about, for example, by a combination of the individual nucleic acid sequences on a polynucleotide (multiple constructs), on a plurality of polynucleotides in a cell (cotransformation) or by sequential transformation are also in accordance with the invention.

Advantageous genetic control sequences for the expression cassettes according to the invention or for vectors comprising them are, for example, promoters such as the cos, tac, trp, tet, lpp, lac, lacIq, T7, T5, T3, gal, trc, ara, SP6, λ-PR or the λ-PL promoter, all of which can be used for expressing a mevalonate kinase, preferably MEK, in Gram-negative bacterial strains.

Examples of further advantageous genetic control sequences are present, for example, in the promoters amy and SPO2, both of which can be used for expressing SSP in Gram-positive bacterial strains, and in the yeast or fungal promoters AUG1, GPD-1, PX6, TEF, CUP1, PGK, GAP1, TPI, PHO5, AOX1, GAL10/CYC1, CYC1, OliC, ADH, TDH, Kex2, MFA or NMT or combinations of the abovementioned promoters (Degryse et al., Yeast 1995 June 15; 11(7):629-40; Romanos et al. Yeast 1992 June;8(6):423-88; Benito et al. Eur. J. Plant Pathol. 104, 207-220 (1998); Cregg et al. Biotechnology (N Y) 1993 August; 11 (8):905-10; Luo X., Gene 1995 Sep. 22;163(1):127-31: Nacken et al., Gene 1996 Oct. 10;175(1-2): 253-60; Turgeon et al., Mol Cell Biol 1987 September;7(9):3297-305) or the transcription terminators NMT, Gcyl, TrpC, AOX1, nos, PGK or CYC1 (Degryse et al., Yeast 1995 June 15; 11 (7):629-40; Brunelli et al. Yeast 1993 (Dec9(12): 1309-18; Frisch et al., Plant Mol. Biol. 27(2), 405-409 (1995); Scorer et al., Biotechnology (N.Y. 12 (2), 181-184 (1994), Genbank acc. number Z46232; Zhao et al. Genbank acc number: AF049064; Punt et al., (1987) Gene 56 (1), 117-124), all of which can be used for expressing SSP in yeast strains.

Examples of genetic control elements which are suitable for expression in insect cells are the polyhedrin promoter and the p10 promoter (Luckow, V. A. and Summers, M. D. (1988) Bio/Techn. 6, 47-55) and, if appropriate, also suitable terminators known to the skilled worker.

Advantageous genetic control sequences for expressing MEK in cell culture, in addition to polyadenylation sequences, are, for example, eukaryotic promoters of viral origin such as, for example, promoters of the polyoma virus, adenovirus 2, cytomegalovirus, HIV thymidine kinase or simian virus 40 and, if appropriate, also suitable terminators known to the skilled worker.

Additional functional elements b) are understood as meaning, by way of example but not of limitation, reporter genes, replication origins, selection markers and what are known as affinity tags, in fusion with the nucleic acid sequence in accordance with the invention, directly or by means of a linker optionally comprising a protease cleavage site. Particularly preferred as further suitable additional functional elements are sequences which ensure that the product is targeted into the vacuole, the mitochondrion, the peroxisome, the endoplasmic reticulum (ER) or, owing to the absence of such operative sequences, remains in the compartment where it is formed, the cytosol (Kermode, Crit. Rev. Plant Sci. 15, 4 (1996), 285-423).

Also in accordance with the invention are vectors comprising at least one copy of the nucleic acid sequences according to the invention and/or the expression cassettes according to the invention.

In addition to plasmids, vectors are furthermore also understood as meaning all of the other known vectors with which the skilled worker is familiar, such as, for example, phages, viruses such as SV40, CMV, baculovirus, adenovirus, transposons, IS elements, phasmids, phagemids, cosmids or linear or circular DNA. These vectors can be replicated autonomously in the host organism or replicated chromosomally; chromosomal replication is preferred.

In a further embodiment of the vector, the nucleic acid construct according to the invention can advantageously also be introduced into the organisms in the form of a linear DNA and integrated into the genome of the host organism via heterologous or homologous recombination. This linear DNA may consist of a linearized plasmid or only of the nucleic acid construct as vector, or the nucleic acid sequences used.

Further prokaryotic or eukaryotic expression systems are mentioned in Chapters 16 and 17 in Sambrook et al., “Molecular Cloning: A Laboratory Manual.” 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The expression cassette according to the invention and vectors derived therefrom can be used for transforming bacteria, cyanobacteria, yeasts, filamentous fungi and algae and eukaryotic nonhuman cells (for example insect cells) with the aim of producing mevalonate kinase, preferably MEK, recombinantly, the generation of a suitable expression cassette depending on the organism in which the gene is to be expressed.

In a further advantageous embodiment, the nucleic acid sequences used in the method according to the invention may also be introduced into an organism by themselves.

If, in addition to the nucleic acid sequences, further genes are to be introduced into the organism, they can all be introduced into the organism together in a single vector, or each individual gene can be introduced into the organism in each case in one vector, it being possible to introduce the different vectors simultaneously or in succession.

In this context, the introduction, into the organisms in question (transformation), of the nucleic acid(s) according to the invention, of the expression cassette or of the vector can be effected in principle by all methods with which the skilled worker is familiar.

In the case of microorganisms, the skilled worker will find suitable transformation methods in the textbooks by Sambrook, J. et al. (1989) “Molecular cloning: A laboratory manual”, Cold Spring Harbor Laboratory Press, by F. M. Ausubel et al. (1994) “Current protocols in molecular biology”, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Habor Laboratory Press or Guthrie et al. “Guide to Yeast Genetics and Molecular Biology”, Methods in Enzymology, 1994, Academic Press.

In the transformation of filamentous fungi, the methods of choice are firstly the generation of protoplasts and transformation with the aid of PEG (Wiebe et al. (1997) Mycol. Res. 101 (7): 971-877; Proctor et al. (1997) Microbiol. 143, 2538-2591), and secondly the transformation with the aid of Agrobacterium tumefaciens (de Groot et al. (1998) Nat. Biotech. 16, 839-842).

The transgenic organisms generated by transformation with one of the above-described embodiments of an expression cassette or of a vector comprise a nucleic acid sequence which encodes a protein with the enzymatic, preferably biological, activity of a mevalonate kinase and
a) a nucleic acid sequence with the sequence shown in SEQ ID NO:3, or
b) a nucleic acid sequence which, owing to the degeneracy of the genetic code, can be deduced by backtranslation of the amino acid sequences shown in SEQ ID NO:4, or
c) functional equivalents of SEQ ID NO:3 with at least 70% identity with SEQ ID NO:3,
d) a nucleic acid sequence which, owing to the degeneracy of the genetic code, can be deduced by backtranslation of the amino acid sequence of a functional equivalent of SEQ ID NO:4 which has at least 80% identity with SEQ ID NO:4;
   and the transgenic organisms comprising a nucleic acid sequence which encodes a protein with the enzymatic, preferably biological, activity of a mevalonate kinase comprising
a) a nucleic acid sequence with the sequence shown in SEQ ID NO:5; or
b) a nucleic acid sequence which, owing to the degeneracy of the genetic code, can be deduced by backtranslation of the amino acid sequence shown in SEQ ID NO:6; or
   c) a functional equivalent of SEQ ID NO:5 with at least 67% identity with SEQ ID NO:5;
   d) a functional equivalent of SEQ ID NO:5 which, owing to the degeneracy of the genetic code, can be deduced by backtranslation of the amino acid sequence of a functional equivalent of SEQ ID NO:6 which has at least 72% identity with SEQ ID NO:6.
      and the recombinant MEK which is obtainable from the abovementioned transgenic organism by means of expression are also subject matter of the present invention.

Other suitable organisms for the recombinant expression of MEK, in addition to bacteria, yeasts, mosses, algae and fungi, are eukaryotic cell lines, preferably bacteria, yeasts and fungi.

Preferred within the bacteria are bacteria of the genus Escherichia, Erwinia, Flavobacterium or Alcaligenes or Cyanobacteria, for example of the genus Synechocystis or Anabena.

Preferred yeasts are yeasts of the genera Saccharomyces, Schizosaccharomyces or Pichia.

Preferred fungi are Aspergillus, Trichoderma, Ashbya, Neurospora, Fusarium, Beauveria, Mortierella, Saprolegnia, Pythium, or other fungi described in Indian Chem Engr. Section B. Vol 37, No 1,2 (1995).

In principle, transgenic animals are also suitable as host organisms, for example C. elegans.

Preferred is also the use of expression systems and vectors which are available to the public or commercially available.

Those which must be mentioned for use in E. coli bacteria are the typical advantageous commercially available fusion and expression vectors pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which comprises glutathione S-transferase (GST), maltose binding protein, or protein A, the pTrc vectors (Amann et al., (1988) Gene 69:301-315), “pKK233-2” by CLONTECH, Palo Alto, Calif., and the “pET” and “pBAD” vector series from Stratagene, La Jolla.

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, pGAPZ derivatives, pPICZ derivatives and the vectors of the “Pichia Expression Kit” (Invitrogen Corporation, San Diego, Calif.). Vectors for use in filamentous fungi are described in: van den Hondel, C. A. M. J. J. & Punt, P. J. (1991) “Gene transfer systems and Vector development for filamentous fungi”, in: Applied Molecular Genetics of Fungi, J. F. Peberdy, et al., eds., pp. 1-28, Cambridge University Press: Cambridge.

As an alternative, insect cell expression vectors may also be used advantageously, for example for expression in Sf9, Sf21 or Hi5 cells, which are infected via recombinant baculoviruses. Examples of these are the vectors of the pAc series (Smith et al. (1983) Mol. Cell Biol. 3:2156-2165) and the pVL series (Lucklow and Summers (1989) Virology 170:31-39). Others which may be mentioned are the Baculorvirus expression systems “MaxBac 2.0 Kit” and “Insect Select System” by Invitrogen, Calsbald or “BacPAK Baculovirus Expression system” by CLONTECH, Palo Alto, Calif. The skilled worker is familiar with the handling of cultured insect cells and with their infection for expressing proteins, which can be carried out analogously to known methods (Luckow and Summers, Bio/Tech. 6,1988, pp. 47-55; Glover and Hames (eds.) in DNA Cloning 2, A practical Approach, Expression Systems, Second Edition, Oxford University Press, 1995, 205-244).

Plant cells or algal cells are others which can be used advantageously for expressing genes. Examples of plant expression vectors can be found in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Moreover, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used in this context are of viral origin such as, for example, promoters of polyoma virus, adenovirus 2, cytomegalovirus or simian virus 40. Further prokaryotic and eukaryotic expression systems are mentioned in Chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

All of the above-described embodiments of possible embodiments of transgenic organisms comprising a nucleic acid sequence according to the invention, for example by transformation with an expression cassette according to the invention, or of a vector comprising an expression cassette according to the invention, are referred to as “organisms according to the invention” hereinbelow.

The present invention furthermore relates to the use of mevalonate kinase, preferably MEK, in a method for identifying fungicidally active test compounds. All methods for identifying fungicidally active inhibitors are hereinbelow referred to as methods according to the invention.

In this context, the method for identifying fungicidally active substances preferably consists of an inhibition assay in which a polypeptide with the enzymatic activity of a mevalonate kinase is used.

A preferred embodiment of the method according to the invention comprises the following steps:
i. bringing the mevalonate kinase, preferably MEK, into contact with one or more test substances under conditions which permit binding of the test substance(s) to the nucleic acid molecule or the polypeptide which is encoded via the abovementioned nucleic acid molecule, and
ii. detecting whether the test substance binds to the polypeptide of i), or
iii. detecting whether the test substances reduce or block the activity of the polypeptide of i), or
iv. detecting whether the test substances reduce or block the transcription, translation or expression of the polypeptide of i).

The detection in accordance with step ii of the above method can be effected using techniques which detect the interaction between protein and ligand. In this context, either the test compound or the enzyme can contain a detectable label such as, for example, a fluorescent label, a radioisotope, a chemiluminescent label or an enzyme label. Examples of enzyme labels are horseradish peroxidase, alkaline phosphatase or luciferase. The subsequent detection depends on the label and is known to the skilled worker.

In this context, five preferred embodiments which are also suitable for high-throughput screening methods (HTS) in connection with the present invention must be mentioned in particular:
1. The average diffusion rate of a fluorescent molecule as a function of the mass can be determined in a small sample volume via fluorescence correlation spectroscopy (FCS) (Proc. Natl. Acad. Sci. USA (1994) 11753-11575). FCS can be employed for determining protein/inhibitor interactions by measuring the changes in the mass, or the changed diffusion rate which this entails, of a test compound when binding to MEK. A method according to the invention can be designed directly for measuring the binding of a test compound labeled by a fluorescent molecule. As an alternative, the method according to the invention can be designed in such a way that a chemical reference compound which is labeled by a fluorescent molecule is displaced by further chemical test compounds (“displacement assay”).
2. Fluoresence polarization exploits the characteristic of a quiescent fluorophore excited with polarized light to likewise emit polarized light. If, however, the fluorophore is allowed to rotate during the excited state, the polarization of the fluorescent light which is emitted is more or less lost. Under otherwise identical conditions (for example temperature, viscosity, solvent), the rotation is a function of molecule size, whereby findings regarding the size of the fluorophore-bound residue can be obtained via the reading (Methods in Enzymology 246 (1995), pp. 283-300). A method according to the invention can be designed directly for measuring the binding of a fluorescently labeled test compound to MEK. As an alternative, the method according to the invention may also take the form of the “displacement assay” described under 1.
3. Fluorescence resonance energy transfer (FRET) is based on the radiation-free energy transfer between two spatially adjacent fluorescent molecules under suitable conditions. A prerequisite is that the emission spectrum of the donor molecule overlaps with the excitation spectrum of the acceptor molecule. By fluorescent labeling of MEK and the test compounds, the binding can be measured by means of FRET (Cytometry 34, 1998, pp. 159-179). As an alternative, the method according to the invention may also take the form of the “displacement assay” described under 1. An especially suitable embodiment of FRET technology is “Homogeneous Time Resolved Fluorescence” (HTRF) as can be obtained from Packard BioScience. The compounds which are identified in this manner may be suitable as inhibitors.
4. Surface-enhanced laser desorption/ionization (SELDI) in combination with a time-of-flight mass spectrometer (MALDI-TOF) makes possible the rapid analysis of molecules on a support and can be used for analyzing protein/ligand interactions (Worral et al., (1998) Anal. Biochem. 70:750-756). In a preferred embodiment, MEK is immobilized on a suitable support and incubated with the chemical compound to be studied. After one or more suitable wash steps, the molecules of the chemical compound which are additionally bound to the mevalonate kinase, preferably MEK, can be detected by means of the above-mentioned methodology and suitable inhibitors can thus be selected.
5. The measurement of surface plasmon resonance is based on the change in the refractive index at a surface when a chemical compound binds to a protein which is immobilized on said surface. Since the change in the refractive index is identical for virtually all proteins and polypeptides for a defined change in the mass concentration at the surface, this method can be applied to any protein in principle (Lindberg et al. Sensor Actuators 4 (1983) 299-304; Malmquist Nature 361 (1993) 186-187). The measurement can be carried out for example with the automatic analyzer based on surface plasmon resonance which is available from Biacore (Freiburg) at a throughput of, currently, up to 384 samples per day. A method according to the invention can be designed directly for measuring the binding of the test compound to MEK. As an alternative, the method according to the invention may also take the form of the “displacement assay” described under 1.

All of the substances identified via the abovementioned methods can subsequently be checked for their fungicidal action in another embodiment of the method according to the invention.

Furthermore, there exists the possibility of detecting further candidates for fungicidal active ingredients by molecular modeling via elucidation of the three-dimensional structure of MEK by x-ray structure analysis. The preparation of protein crystals required for x-ray structure analysis, and the relevant measurements and subsequent evaluations of these measurements, the detection of a binding site in the protein, and the prediction of potential inhibitor structures are known to the skilled worker. In principle, an optimization of the compounds identified by the abovementioned methods is also possible via molecular modeling.

A preferred embodiment of the method according to the invention, which is based on steps i) and iii), consists in
a) either expressing mevalonate kinase, preferably MEK, in an organism or culturing an organism which naturally contains mevalonate kinase, preferably MEK;
   b) bringing mevalonate kinase, preferably MEK, of step a) in the cell digest of the transgenic or nontransgenic organism, in partially purified form or in homogeneously purified form, into contact with a test compound; and
   c) selecting a compound which reduces or blocks the mevalonate kinase, preferably MEK, activity, the activity of the mevalonate kinase, preferably MEK, incubated with the test compound being compared with the activity of a mevalonate kinase, preferably MEK, not incubated with a test compound.

In this step (c), compounds are selected which bring about a significant decrease in the activity of mevalonate kinase, preferably MEK, in comparison with a mevalonate kinase, preferably MEK, which has not been incubated with a chemical compound, achieving a reduction by at least 10%, advantageously at least 20%, preferably at least 30%, particularly preferably by at least 50% and very particularly preferably by at least 70%, or 100% reduction (blocking).

The solution containing the mevalonate kinase, preferably MEK, can consist of the lysate of the original organism or of the transgenic organism. If necessary, the mevalonate kinase, preferably MEK, can be purified partially or fully via customary methods. A general overview of current protein purification techniques is described, for example, in Ausubel, F. M. et al., Current Protocols in Molecular Biology, Greene Publishing Assoc. and Wiley-Interscience (1994); ISBN 0-87969-309-6. If obtained recombinantly, the protein which takes the form of a fusion with an affinity tag can be purified via affinity chromatography.

The mevalonate kinase, preferably MEK, which is required for in vitro methods can thus be isolated either by means of heterologous expression from a transgenic organism according to the invention, or mevalonate kinase, preferably MEK, can be isolated from an organism comprising mevalonate kinase, preferably MEK, for example from a fungus or a yeast (see, for example: Imblum and Rodwell (1975) J. Lipid Res., 15, 211-222). Suitable yeasts can be found within the genera Saccharomyces, Schizosaccharomyces or Pichia. Suitable yeasts and filamentous fungi are the species mentioned at the outset.

The determination of the activity of mevalonate kinase, preferably MEK, can be effected for example via an enzyme activity assay, i.e. by incubating the polypeptide according to the invention with a suitable substrate, the decrease of the substrate, or increase of the forming product, or the decrease or increase of the cofactor being monitored.

Examples of suitable substrates are, for example, mevalonate, and examples of suitable cofactors are ATP, GTP or UTP, preferably ATP, and Mg²⁺ or Mn²⁺, preferably Mn²⁺. If appropriate, derivatives of the abovementioned compounds which contain a detectable marker may also be used, such as, for example, a fluorescent label, a radioisotope label (for example ¹⁴C-mevalonate, γ³² or γ33 ATP) or a chemiluminescent label.

The amounts of substrate to be employed in the activity assay may range from 0.5-100 mM and the amounts of cofactor from 0.1-5 mM, based on 1-100 μg/ml enzyme.

In a particularly preferred embodiment, the conversion of the substrate is monitored photometrically. For example, reference may be made here to the assay described by Porter J. B. (1985; Meth. Enzymol. 110, 71-79), which is based on coupling the mevalonate kinase reaction with the reaction catalyzed by pyruvate kinase and lactate dehydrogenase, where the oxidation of NADH is a measure of the activity of mevalonate kinase. In slightly modified form, such as described by Schulte et al. (1999; Anal. Biochem. 269, 245-54), this assay is also suitable for high-throughput methods.

A preferred embodiment of the method according to the invention, which is based on steps i) and iv), consists of the following steps:
i. generating a transgenic organism according to the invention,
ii. applying a test substance to the transgenic organism of step i and to a nontransgenic organism of the same species,
iii. determining the growth, the viability and/or the infectivity of the transgenic organism and of the nontransgenic organism after application of the test substance, and
iv. selecting test substances which bring about reduced growth, viability and/or infectivity of the nontransgenic organism in comparison with the growth of the transgenic organism.

In this context, the difference in growth, or the difference with regard to the infectivity, in step iv) for the selection of a fungicidally active inhibitor amounts to at least 10%, by preference 20%, preferably 30%, especially preferably 40% and very especially preferably 50%. The infectivity is only determined when the organism is a phytopathogenic fungus.

As mentioned above, the transgenic organism can be generated by transforming the organism with a nucleic acid sequence according to the invention, an expression cassette according to the invention or a vector comprising a nucleic acid sequence according to the invention or an expression cassette according to the invention.

In this context, the transgenic cells or organisms are bacteria, yeasts, filamentous fungi or eukaryotic cell lines, preferably phytopathogenic filamentous fungi, especially preferably the phytopathogenic filamentous fungi mentioned on pages 10 and 11. These transgenic organisms or cells thus show increased tolerance to chemical compounds which inhibit the polypeptide according to the invention.

All of the compounds which have been identified via the abovementioned methods can subsequently be tested in vivo for their fungicidal action in a further activity assay. One possibility consists in testing the substance in question in agar diffusion tests as described by Zähner, H. 1965 Biologie der Antibiotika, Berlin, Springer Verlag. The test is carried out using a culture of a filamentous fungus, preferably a culture of a filamentous phytopathogenic fungus, it being possible to observe the fungicidal activity, for example, via limited growth. A phytopathogenic fungus is to be understood here as meaning the species mentioned at the outset.

It is also possible, in the method according to the invention, to employ a plurality of test compounds in a method according to the invention. If a group of test compounds affect the target, then it is either possible directly to isolate the individual test compounds or to divide the group of test compounds into a variety of subgroups, for example when it consists of a multiplicity of different components, in order to reduce the number of different test compounds in the method according to the invention. The method according to the invention is then repeated with the individual test compound or with the corresponding subgroup of test compounds. Depending on the complexity of the sample, the above-described steps can be carried out repeatedly, preferably until the subgroup identified in accordance with the method according to the invention only comprises a small number of test compounds, or indeed just one test compound.

The method according to the invention can advantageously-also be carried out as a high-throughput method, or high-throughput screen (HTS), since this enables the parallel testing of a multiplicity of different compounds.

The use of supports which contain one or more of the nucleic acid molecules according to the invention, one or more vectors containing the nucleic acid sequence according to the invention, one or more transgenic organisms which at least one of the nucleic acid sequences according to the invention or one or more (poly)peptides encoded via the nucleic acid sequences according to the invention lends itself to carrying out an HTS in practice. The support used can be solid or liquid; it is preferably solid and especially preferably a microtiter plate. The abovementioned supports are also subject matter of the present invention. In accordance with the most widely used technique, 96-well microtiter plates which, as a rule, can comprise volumes of 50-500 μl are used. Besides the microtiter plates, the further components of an HTS system which match the corresponding microtiter plates, such as a large number of instruments, materials, automatic pipetting devices, robots, automated plate readers and plate washers, are commercially available.

In addition to the HTS systems based on microtiter plates, what are known as “free-format assays” or assay systems where no physical barriers exist between the samples such as, for example, in Jayaickreme et al., Proc. Natl. Acad. Sci. U.S.A. 19 (1994) 161418; Chelsky, “Strategies for Screening Combinatorial Libraries”, First Annual Conference of The Society for Biomolecular Screening in Philadelphia, Pa. (Nov. 7-10,1995); Salmon et al., Molecular Diversity 2 (1996), 5763 and U.S. Pat. No. 5,976,813, may also be used.

The invention furthermore relates to compounds identified by the methods according to the invention. These compounds are hereinbelow referred to as “selected compounds”. They have a molecular weight of less than 1000 g/mol, advantageously less than 500 g/mol, preferably less than 400 g/mol, especially preferably less than 300 g/mol. Herbicidally active compounds have a Ki value of less than 1 mM, preferably less than 1 μM, especially preferably less than 0.1 μM, very especially preferably less than 0.01 μM.

The selected compounds are suitable for controlling phytopathogenic fungi. Examples of phytopathogenic fungi are the abovementioned genera and species.

The selected compounds can also be present in the form of their agriculturally useful salts. Agriculturally useful salts are mainly the salts of those cations or the acid addition salts of those acids whose cations, or anions, do not adversely affect the fungicidal activity of the fungicidally active compounds identified via the methods according to the invention.

If chiral centers are present, all of the compounds identified via the above-mentioned methods, not only as pure enantiomers or diastereomers, but also as their mixtures or as a racemate, are subject matter of the present invention.

The selected compounds can be chemically synthesized substances or substances produced by microorganisms and can be found, for example, in cell extracts of, for example, plants, animals or microorganisms. The reaction mixture can be a cell-free extract or comprise a cell or cell culture. Suitable methods are known to the skilled worker and are described generally for example in Alberts, Molecular Biology the cell, 3^{rd} Edition (1994), for example chapter 17.

Candidate test-compounds can be expression libraries such as, for example, cDNA expression libraries, peptides, proteins, nucleic acids, antibodies, small organic substances, hormones, PNAs or the like (Milner, Nature Medicine 1 (1995), 879-880; Hupp, Cell. 83 (1995), 237-245; Gibbs, Cell. 79 (1994), 193-198 and references cited therein).

Fungicidal compositions comprising the selected compounds effect very good control of phytopathogenic fungi, in particular when applied at high rates. In crops such as wheat, rice, maize, soya and cotton, they act against phytopathogenic fungi without substantially damaging the crop plants. This effect is observed mainly at low application rates. Whether the fungicidal active ingredients found with the aid of the methods according to the invention act as nonselective or selective fungicides depends, inter alia, on the application rate, their selectivity and other factors. The substances can be used for controlling the phytopathogenic fungi which have already been mentioned above.

Depending on the application method in question, the selected compounds, or compositions comprising them, can be used advantageously for eliminating the phytopathogenic fungi which have already been mentioned at the outset.

The invention furthermore relates to a method for preparing the fungicidal composition which has already been mentioned above, which comprises formulating selected compounds with adjuvants suitable for the formulation of fungicides.

The selected compounds an be formulated for example as directly sprayable aqueous solutions, powders, suspensions, also highly concentrated aqueous, oily or other suspensions or suspoemulsions or dispersions, emulsifiable concentrates, emulsions, oil dispersions, pastes, dusts, materials for spreading or granules, and applied by spraying, fogging, dusting, spreading or pouring. The use forms depend on the intended use and the nature of the selected compounds; in any case, they should ensure as fine as possible a distribution of the selected compounds. The fungidical compositions comprise a fungicidally active amount of at least one selected compound and adjuvants conventionally used for the formulation of fungicidal compositions.

To prepare emulsions, pastes or aqueous or oily formulations and dispersible concentrates (DC), the selected compounds can be dissolved or dispersed in an oil or solvent, it being possible to add further formulation adjuvants for homogenization purposes. However, it is also possible to prepare liquid or solid concentrates from selected compound, if appropriate solvents or oil and, optionally, further adjuvants, and such concentrates are suitable for dilution with water. Formulations which must be mentioned in this context are emulsifiable concentrates (EC, EW), suspensions (SC), soluble concentrates (SL), dispersible concentrates (DC), pastes, pills, wettable powders or granules, it being possible for the solid formulations to be either soluble in water or dispersible in water (wettable). Moreover, suitable powders, granules or tablets may additionally be provided with a solid coating which prevents abrasion or the premature release of active ingredients.

In principle, an adjuvant is understood as meaning the following classes of substances: antifoams, thickeners, wetters, stickers, dispersants, emulsifiers, bactericides and/or thixotropics. The importance of the abovementioned agents is known to the skilled worker.

SLs, EWs and ECs can be prepared by simply mixing the constituents in question; powders can be prepared by mixing or grinding in specific types of mills (for example hammer mills). DC, SCs and SEs are usually prepared by wet milling, it being possible to prepare an SE from an SC by addition of an organic phase, which may comprise further auxiliaries or selected compounds. The preparation is known. Powders, materials for spreading and dusts can advantageously be prepared by mixing or concomitantly grinding the active ingredients together with a solid carrier. Granules, for example coated granules, impregnated granules and homogeneous granules, can be prepared by binding the selected compounds to solid carriers. Further preparation details are known to the skilled worker and detailed for example in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and ff.

WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

A multiplicity of inert liquid and/or solid carriers which are suitable for the formulations according to the invention are known to the skilled worker, such as, for example, liquid additives like mineral oil fractions of medium to high boiling point such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone or strongly polar solvents, for example, amines such as N-methylpyrrolidone, or water.

Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The skilled worker is familiar with the multiplicity of surface-active substances (surfactants) which are suitable for the formulations according to the invention such as, for example, alkali metal salts, alkaline earth metal salts or ammonium salts of aromatic sulfonic acids, for example lignosulfonic acid, phenolsulfonic acid, naphthalenesulfonic acid, and dibutylnaphthalenesulfonic acid, and of fatty acids, of alkyl- and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

The fungicidal compositions, or the active ingredients, can be applied curatively, eradicatively or protectively. Depending on the control target, the season, the target plants and the growth stage, the application rates of the fungicidal actives (substances and/or compositions) amount to 0.001 to 3.0, preferably 0.01 to 1.0 kg/ha.

The invention is illustrated in greater detail by the examples which follow, but is not limited thereto.

The following is a brief description of the recombinant methods on which the following examples are based: Cloning methods such as, for example, restriction cleavages, DNA isolation, agarose gel electrophoresis, purification of DNA fragments, transfer of nucleic acids to nitrocellulose and nylon membranes, linking of DNA fragments, transformation of E. coli cells, growing of bacteria, sequence analysis of recombinant DNA, and Southern and Western blots were carried out as described by Sambrook et al., Cold Spring Harbor Laboratory Press (1989) and Ausubel, F. M. et al., Current Protocols in Molecular Biology, Greene Publishing Assoc. and Wiley-Interscience (1994); ISBN 0-87969-309-6.

The bacterial strains used hereinbelow (E. coli TOP10) were obtained from Invitrogen, Carlsberg, C A. A possible F. graminearum wild-type strain which may be used is the strain DSM:4527.

Moreover, all of the chemicals used hereinbelow were obtained in analytical-grade form from Fluka (Neu-Ulm), Merck (Darmstadt), Roth (Karlsruhe), Serva (Heidelberg) and Sigma (Deisenhofen), unless otherwise specified. Solutions were made with purified, pyrogen-free water, hereinbelow referred to as H₂O, from a Milli-Q Water System water purification system (Millipore, Eschborn). Restriction enzymes, DNA-modifying enzymes and molecular biology kits were obtained from AGS (Heidelberg), Amersham (Braunschweig), Biometra (Gottingen), Roche (Mannheim), Genomed (Bad Oeynnhausen), New England Biolabs (Schwalbach/Taunus), Novagen (Madison, Wis., USA), Perkin-Elmer (Weiterstadt), Promega (Madison, Wis., USA), Pharmacia (Freiburg), Qiagen (Hilden) and Stratagene (Heidelberg). Unless otherwise specified, they were used in accordance with the manufacturers' instructions.

All of the media and buffers used for the recombinant experiments were sterilized either by filter sterilization or by heating in an autoclave.

### EXAMPLES

### Example 1

### Generation of the Vector pUCmini-Hyg

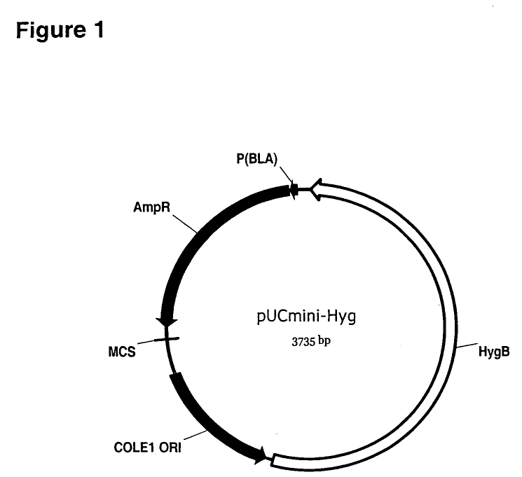

The plasmid pUCmin-Hyg is shown in FIG. 1.

A 2536 bp DNA of the Cochliobolus heterotrophus GPD1 promoter, linked to the E. coli hygromycin B resistance gene, was amplified via PCR using the primers
P 1 5′ atgaagcttggggtttgagggccaatggaacgaaactagtgtaccacttgacc 3′ (SEQ ID NO:7) and
P 2 5′gacagatctggcgccattcgccattcag 3′ (SEQ ID NO:8)
   with pGUS5 as template (M6nke, E. and Schäfer, W., 1993, Mol. Gen. Genet. 241: 73-80). The PCR was carried out following standard conditions (as described in Sambrook, J. et al. (1989) “Molecular cloning: A laboratory manual”, Cold Spring Harbor Laboratory Press).

The DNA fragment obtained in the PCR was cloned into the plasmid pFDX3809 (WO 01/38504) via the restriction enzyme cleavage sites Hind III and Bgl II, which are present in P1 and P2. The resulting plasmid pHygB was used as template in a further PCR, in which the primers

| | | |
|---|---|---|
| P3 | 5′ ggaatcggtcaatacactac 3′ | (SEQ ID NO:9) |
| and | | |
| P4 | 5′ tgtagatctctattcctttgccctcggacgagt 3′ | (SEQ ID NO:10) |

were used for specifically truncating the hygromycin B resistance gene. The resulting DNA fragment, consisting of 575 bp of the 3′ end of the hygromycin B resistance gene, was cloned into plasmid pHygB via the restriction enzyme cleavage sites Nde II/Bgl II, giving rise to the plasmid pHygB-NOS.

A 2019 bp Hind III/Ssp I DNA fragment comprising the expression cassette consisting of GPD1 promoter, hygromycin B resistance gene and nopaline synthase terminator was excised from pHygB-NOS and cloned into the plasmid pFDX3809 (see WO 01/38504) via EcoRI and HindIII, giving rise to the plasmid pUCmini-Hyg. To this end, the EcoRI cleavage sites were made compatible with Ssp I (via fill-in treatment using the DNA polymerase I Klenow fragment).

### Example 2

### Generation of Knock-Out Transformants

A) Generation of the plasmids pUCmini-Hyg-MevKin and pUCmini-Hyg-PKS

To generate the knock-out plasmid for MEK, a 428 bp mevalonate kinase fragment was amplified from F. graminearum with the aid of the primers P5 and P6 (SEQ ID NO:3). cDNA of this fungus acted as the template. To generate the knock-out plasmid for the knock-out control of PKS, a 635 bp fragment was amplified with the aid of primers P7 and P8 (SEQ ID NO:5)

| | |
|---|---|
| P 5: | |
| ataagaatgcggccgcTACTCCAAACCACCCAACGT | (SEQ ID NO:11) |
| P 6: | |
| aaatggcgcgccCTTCTGAAGCTTCTCAGCAG | (SEQ ID NO:12) |
| P 7: | |
| ataagaatgcggccgcAATGGCCCTCGAAACAGC | (SEQ ID NO:13) |
| P 8: | |
| aaatggcgcgccGCGCCCAGAATGACACC | (SEQ ID NO:14) |

By means of the AscI and NotI restriction sites which had been introduced, the fragments were cloned into the vector pUCmini-Hyg, giving rise to the vectors pUCmini-Hyg-MevKin and pUCmini-Hyg-PKS.

Using SEQ ID NO:3, it was possible to identify the full-length clone SEQ ID NO:5 which belongs to SEQ ID NO:3.

B) Protoplast Preparation.

To obtain protoplasts of the F. graminearum WT strain 8/1, mycelium was incubated for 2 days at 28° C. and 180 rpm in CM_{compt} as described by Leach et al. (J. Gen. Microbiol. 128 (1982) 1719-1729) as a liquid culture, comminuted and subsequently incubated for a further day at 28° C., 180 rpm. The mycelium was then washed twice with distilled water. 2 g of mycelium were treated with 20 ml of 5% enzyme osmotic solution (700 mM NaCl, 5% Driselase, sterile) and incubated for 3 hours at 28° C. and 100 rpm. The progressive release of protoplasts was monitored under the microscope using samples. Protoplasts were separated from mycelial debris by filtration, pelleted (3000 rpm, 10 min, 4° C.), and, after washing with in each case 10 ml of 700 mM NaCl and SORB-TC (1.2 M sorbitol, 50 mM CaCl₂, 10 mM Tris/HCl, pH 7.0), taken up in 1 ml of SORB-TC. The protoplast concentration was determined by counting under the microscope.

C) Transformation

For the subsequent transformation of F. graminearum protoplasts, the plasmids were isolated following standard procedures as they are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1994). Thereafter, the plasmid pUCmini-Hyg-MevKin and the plasmid pUCmini-Hyg-PKS were linearized in the middle using EcoNI and Eco47111, respectively.

For the transformation, 10⁷ protoplasts were placed on ice, mixed carefully with 30 μg of the plasmid prepared as described above and subsequently incubated on ice for 10 minutes. After addition of one volume of PEG-TC (60% (w/v) PEG4000, 50 mM CaCl₂, 10 mM Tris/HCl pH 7.0) and subsequent incubation for 15 minutes on ice, 8 volumes—based on the original culture volume—of SORB-TC medium were added. This solution was mixed with 400 ml of regeneration medium at a temperature of 45° C. (1 g/l yeast extract, 1 g/l casein hydrolysate, 342 g/l sucrose, 16 g/l agar), divided into 20-ml portions and placed into the corresponding number of Petri dishes of 90 mm diameter.

D) Selection of the Resulting Knock-Out Transformants

After 1 day's incubation at 28° C., each of the Petri dishes was covered with a layer of 10 ml of hygromycin-containing water agar (16 g/l agar, 300 mg/l hygromycin) and subsequently incubated at 28° C. Mycelial colonies growing through the selection agar were excised and placed individually on CM_{hyg} plates (CM_{compt} medium supplemented with 150 mg/l of hygromycin).

E) Detection of the Knock-Out Transformants

DNA from the mycelium of transformants was verified for integration of the knock-out construct with the aid of PCR. The following primers were used:

| | |
|---|---|
| P 9: | |
| GGGGAGGAAAGGCTGTGGTGTT | (SEQ ID NO:15) |
| P 10: | |
| CGTCTTCCTCGGTGCCGTTCTT | (SEQ ID NO:16) |
| P 11: | |
| ATGTCTCCAAAGGAAGCTGAGC | (SEQ ID NO:17) |
| P 12: | |
| TCGAGTGATGGATACTGCTTCG | (SEQ ID NO:18) |
| P 13: | |
| CGGCTACACTAGAAGGACAGTATTTGGTA | (SEQ ID NO:19) |
| P 14: | |
| GTCAGGCAACTATGGATGAACGAAATAGAC | (SEQ ID NO:20) |

The PCR was carried out using standard conditions (for example as described by Sambrook, J. et al. (1989) “Molecular cloning: A laboratory manual”, Cold Spring Harbor Laboratory Press) in 36 cycles, the first step involving denaturation for 300 seconds at 95° C. and incubation at 72° C. being performed for 600 seconds after 25 cycles (in each case 90 seconds at 95° C. (denaturing); 90 seconds at 55° C. (annealing), 120 seconds at 72° C. (elongation)).

The PCR screening of the transformants obtained was divided into the following steps:

Step 1: Amplification of the gene fragment from genomic DNA. To this end, P 9 and P 10 were used in the case of mevalonate kinase and P 11 and P 12 in the case of PKS. The PCR conditions were chosen in such a way that a 500 bp product was expected only in the case of ectopic integration, but not in the case of homologous recombination.

Step 2: Amplification of a region in which a primer binds to the genomic DNA and another primer to the integrated vector. Owing to the primer combination, an amplificate was obtained in the case of homologous recombination only. This approach enabled the validation of the first step. The primer combinations P 9, P 14 and P 10, P 13 were used in the case of mevalonate kinase, while the primer combinations P 11, P 14 and P 12, P 13 were used in the case of PKS.

F) Result

F. graminearum was transformed in two independent experiments in order to destroy the mevalonate kinase gene with the above-described knock-out plasmid. As the control, the gene of a PKS was destroyed with the aid of the knock-out construct pUCminIV-PKS. All of the tested transformants were studied using the PCR screening described under E. In the approach for destroying the PKS gene, 9 transformants were studied for homologous recombination, which was identified in all of the transformants.

In contrast, all of the studied transformants in which mevalonate kinase was to be destroyed revealed ectopic insertion. It can be seen that transformants in which mevalonate kinase is destroyed are not viable and that the gene is thus essential for the fungus.

### Example 3

In order to produce sufficient amounts of protein, for example for use in HTS, the procedure of choice is the overexpression of the protein in a suitable system. To this end, the cDNA sequence of the mevalonate kinase from N. crassa mRNA can be amplified by means of suitable primers, which are deduced from SEQ ID NO:1, via PCR under standard conditions (for example as described by Sambrook, J. et al. (1989) “Molecular cloning: A laboratory manual”, Cold Spring Harbor Laboratory Press):

| | |
|---|---|
| P 15: | |
| GCA GAG CAA GAA CAC AAC | (SEQ ID NO:21) |
| P 16: | |
| GGC TAC TAG AAG CTT CTA GTC CCG GTT CTC AAC | (SEQ ID NO:22) |
| P 17: | |
| CAC CAT GGC AGA GCA AGA ACA CAA | (SEQ ID NO:23) |
| P 18: | |
| GTC CCG GTT CTC AAC CCG | (SEQ ID NO:24) |

Subsequently, the resulting PCR fragment can be cloned into suitable vectors such as, for example, pMALc2x (P 15 and P 16) or pET101-D/TOPO(P 17 and P 18) in order to produce an N-terminal (MBP, pMALc2x) or C-terminal fusion protein (His6, pET101-D/TOPO). Protein overexpression is carried out with the aid of E. coli BL21 cells. Thereafter, the protein can be purified for use in the activity assays, for example as described in example 4, using affinity chromatography over suitable columns.

### Example 4

### Activity Assays

Fungicidally active compounds which reduce or block the activity of mevalonate kinase are selected by comparing the activity of the mevalonate kinase which has been incubated with the test compound with the activity of a mevalonate kinase which has not been incubated with a test compound, it being possible to determine the activity as described in example 4A) or B).

A) Spectrophotometric Assay

Mevalonate kinase phosphorylates mevalonate using ATP, giving rise to phosphomevalonate and ADP. In order to search for inhibitors of this enzyme, the ADP which is formed can be detected by the coupled reactions with the enzymes pyruvate kinase and lactate dehydrogenase. What is measured is, ultimately, the oxidation of NADH at 340 nm. The reaction mixture contains the following in a total volume of one milliliter: KH₂PO₄ (100 mM, pH 7.0), 2-mercaptoethanol or dithiotreithol (10 mM), NADH (0.16 mM), MgCl₂ (5 mM), MgATP (4 mM), DL-mevalonate (3 mM), mevalonate kinase (approx. 0.01 U), phosphoenol pyruvate (0.5 mM), lactate dehydrogenase (0.05 mg protein and 27 U) and pyruvate kinase (0.05 mg protein and 20 U). The reaction is started by adding mevalonate kinase (Porter J. B. (1985) Meth. Enzymol. 110, 71-79). A somewhat modified form of the test can also be carried out in microtiter plate format (Schulte et al. (1999) Anal. Biochem. 269, 245-54).

B) Assay with Radioactive Chemicals

What is measured in this assay is the amount of phosphorylated derivative of DL-[2-¹⁴C] mevalonate by separating the reaction mixture by means of thin-layer chromatography and subsequently measuring the radioactivity of the band in question. The reaction mixture consists of KH₂PO₄ (100 mM, pH 7.0), 2-mercaptoethanol (10 mM), MgCl₂ (5 mM), ATP (4 mM), DL-[2-¹⁴C] mevalonate (3 mM), mevalonate kinase (approx. 0.01 U) in a small volume (0.1-0.2 ml). After the incubation, the reaction is quenched by boiling, the mixture is centrifuged and all the supernatant is applied to Whatman No.1 paper. The chromatogram is developed for 12 hours in a solvent mixture of 1-propanol: ammonia: water (60:20:10). The paper is scanned for radioactivity, and the 5-phosphomevalonate band is excised and measured in a scintillation counter (Porter J. B. (1985) Meth. Enzymol. 110, 71-79).

Explanations for the Sequence Listing

| | | |
|---|---|---|
| SEQ ID NO:1 | NC* | NA*** |
| SEQ ID NO:2 | NC | AA**** |
| SEQ ID NO:3 | FG** | NA |
| SEQ ID NO:4 | FG | AA |
| SEQ ID NO:5 | FG | NA |
| SEQ ID NO:6 | FG | AA |
| SEQ ID NO:7-24 | primer sequences | NA |

*NC Neurospora crassa
**FG Fusarium graminearum
***NA nucleic acid sequence
****AA amino acid sequence

1. The use of A target for fungicides comprising a polypeptide with the enzymatic activity of a mevalonate kinase, the polypeptide encoded by a nucleic acid sequence comprising
a) a nucleic acid sequence with the sequence shown in SEQ ID NO:1 or 5, or
b) a nucleic acid sequence which, owing to the degeneracy of the genetic code, can be deduced by backtranslation of the amino acid sequences shown in SEQ ID NO:2 or 6, or
c) a nucleic acid sequence which, owing to the degeneracy of the genetic code, can be deduced by backtranslation of the amino acid sequence of a functional equivalent of SEQ ID NO:2 which has at least 35% identity with SEQ ID NO:2, or
d) a nucleic acid sequence which, owing to the degeneracy of the genetic code, can be deduced by backtranslation of the amino acid sequence of a functional equivalent of SEQ ID NO:6 which has at least 35% identity with SEQ ID NO:6.

2. The target of claim 1, wherein the nucleic acid sequences encoding a polypeptide with the enzymatic activity of a mevalonate kinase are derived from a filamentous fungus.

3. A nucleic acid encoding a polypeptide with the biological activity of a mevalonate kinase comprising
a) a nucleic acid sequence with the sequence shown in SEQ ID NO:5, or
b) a nucleic acid sequence which, owing to the degeneracy of the genetic code, can be deduced by backtranslation of the amino acid sequences shown in SEQ ID NO:6, or
c) functional equivalents of SEQ ID NO:5 with at least 67% identity with SEQ ID NO:5, or
d) a functional equivalent of SEQ ID NO:5 which, owing to the degeneracy of the genetic code, can be deduced by backtranslation of the amino acid sequence of a functional equivalent of SEQ ID NO:6 which has at least 72% identity with SEQ ID NO:6.

4. The nucleic acid as claimed in claim 3 which is derived from a filamentous fungus.

5. A polypeptide encoded by the nucleic acid molecule of claim 3.

6. A method for detecting functional analogs of SEQ ID NO:1 or SEQ ID NO:3 comprising generating a probe followed by subsequently screening a genomic library or cDNA library of the species in question or conducting a computer search for analogous sequences in electronic databases.

7. A method for identifying mutations in a nucleic acid sequence encoding a polypeptide with the enzymatic activity of a mevalonate kinase derived from a fungus, consisting of
i. generating oligonucleotides based on a nucleic acid sequence of claim 3 comprising the mutation, followed by PCR, or
ii. generating oligonucleotides based on a nucleic acid sequence of claim 3, the mutation-flanking region being amplified by means of PCR, followed by a restriction digest and/or by sequencing of the resulting PCR product.

8. An expression cassette comprising
a) genetic control sequences in operable linkage with the nucleic acid of claim 3.

9. A vector comprising an expression cassette as claimed in claim 8.

10. A nonhuman transgenic organism comprising:
(a) at least one nucleic acid encoding a polypeptide with the biological activity of a mevalonate kinase comprising
i) a nucleic acid sequence with the sequence shown in SEQ ID NO:5, or
ii) a nucleic acid sequence which, owing to the degeneracy of the genetic code, can be deduced by backtranslation of the amino acid sequences shown in SEQ ID NO:6, or
iii) functional equivalents of SEQ ID NO:5 with at least 67% identity with SEQ ID NO:5, or
iv) a functional equivalent of SEQ ID NO:5 which, owing to the degeneracy of the genetic code, can be deduced by backtranslation of the amino acid sequence of a functional equivalent of SEQ ID NO:6 which has at least 72% identity with SEQ ID NO:6,
(b) an expression cassette comprising genetic control sequences in operable linkage with a nucleic acid of (a), or
(c) a vector comprising an expression cassette of (b), selected from among bacteria, yeasts, fungi and animal or plant cells.

11. A method for identifying fungicidally active substances comprising conducting an inhibition test using a polypeptide with the enzymatic activity of a mevalonate kinase.

12. The method as claimed in claim 11, wherein the polypeptide with the enzymatic activity of a mevalonate kinase is encoded by a nucleic acid comprising
a) a nucleic acid sequence encoding a polypeptide with the biological activity of a mevalonate kinase comprising
i) a nucleic acid sequence with the sequence shown in SEQ ID NO:5, or
ii) a nucleic acid sequence which, owing to the degeneracy of the genetic code, can be deduced by backtranslation of the amino acid sequences shown in SEQ ID NO:6, or
iii) functional equivalents of SEQ ID NO:5 with at least 67% identity with SEQ ID NO:5, or
iv) a functional equivalent of SEQ ID NO:5 which, owing to the degeneracy of the genetic code, can be deduced by backtranslation of the amino acid sequence of a functional equivalent of SEQ ID NO:6 which has at least 72% identity with SEQ ID NO:6,
b) a nucleic acid sequence with the sequence shown in SEQ ID NO:1 or SEQ ID NO:5,
c) a nucleic acid sequence which, owing to the degeneracy of the genetic code, can be deduced by backtranslation of the amino acid sequences shown in SEQ ID NO:2 or SEQ ID NO:6,
d) a nucleic acid sequence which, owing to the degeneracy of the genetic code, can be deduced by backtranslation of the amino acid sequence of a functional equivalent of SEQ ID NO:2 which has at least 35% identity with SEQ ID NO:2, or
e) a nucleic acid sequence which, owing to the degeneracy of the genetic code, can be deduced by backtranslation of the amino acid sequence of a functional equivalent of SEQ ID NO:6 which has at least 35% identity with SEQ ID NO:6.

13. The method as claimed in claim 12, wherein the nucleic acid encoding mevalonate kinase is derived from a filamentous fungus.

14. The A method of claim 11 comprising
i. bringing a polypeptide with the enzymatic activity of a mevalonate kinase into contact with one or more test substances under conditions which permit the binding of the test substance(s) to the polypeptide or a nucleic acid molecule encoding the polypeptide, and
ii. detecting whether the test compound binds to the polypeptide of i),
iii. detecting whether the test compound reduces or blocks the activity of the polypeptide of i), or
iv. detecting whether the test compound reduces or blocks the transcription, translation or expression of the nucleic acid of i).

15. The method as claimed in claim 14, wherein
i. either the polypeptide with the enzymatic activity of a mevalonate kinase is expressed in a transgenic organism or an organism which naturally contains mevalonate kinase is cultured,
ii. the polypeptide of step i) in a cell digest of the transgenic or nontransgenic organism, in partially purified form or in homogeneously purified form, is brought into contact with a test compound, and
iii. a compound which reduces or blocks the enzymatic activity of the polypeptide is selected, the enzymatic activity of the polypeptide incubated with the test compound being determined with the enzymatic activity of a polypeptide not incubated with a test compound.

16. The method as claimed in claim 14, which comprises the following steps:
i. culturing a transgenic organism as claimed in claim 10 or a transgenic organism comprising a nucleic acid comprising
a) a nucleic acid sequence with the sequence shown in SEQ ID NO:1 or SEQ ID NO:5,
b) a nucleic acid sequence which, owing to the degeneracy of the genetic code, can be deduced by backtranslation of the amino acid sequences shown in SEQ ID NO:2 or SEQ ID NO:6,
c) a nucleic acid sequence which, owing to the degeneracy of the genetic code, can be deduced by backtranslation of the amino acid sequence of a functional equivalent of SEQ ID NO:2 which has at least 35% identity with SEQ ID NO:2, or
d) a nucleic acid sequence which, owing to the degeneracy of the genetic code, can be deduced by backtranslation of the amino acid sequence of a functional equivalent of SEQ ID NO:6 which has at least 35% identity with SEQ ID NO:6;
ii. applying a test substance to the transgenic organism of step i) and to a nontransgenic organism of the same species,
iii. determining the growth, the viability and/or the infectivity of the transgenic organism and of the nontransgenic organism after application of the test substance, and
iv. selecting test substances which bring about reduced growth, viability and/or infectivity of the nontransgenic organism in comparison with the growth of the transgenic organism.

17. The method of 16, which is carried out using a fungus.

18. The method of claim 11, wherein the substances are identified in a high-throughput screening.

19. A support comprising one or more of the nucleic acid molecules of 3, one or more vectors as claimed in claim 9, one or more transgenic organisms as claimed in claim 10 or one or more (poly)peptide(s) as claimed in claim 5.

20. A fungicidally active compound identified via the method of claim 11.

21. A method for preparing a fungicidal composition, which comprises formulating a fungicidal active ingredient identifiable via the method of claim 11 together with adjuvants which are suitable for the formulation of fungicides.

22. A method of controlling harmful fungi, which comprises treating the fungi or the materials, plants, soils or seeds to be protected from fungal infection with an effective amount of a compound as claimed in claim 20 or a composition which can be prepared by a method as claimed in claim 21.
